(19) United States
(12) Patent Application Publication
(10) Pub. No.: US 2026/0256449 A1
(43) Pub. Date: Sep. 3, 2026

(54) SYSTEMS AND METHODS FOR AI ASSISTED ECHOCARDIOGRAPHY

(71) Applicant: ECHOIQ LIMITED, Sydney, New South Wales (AU)

(72) Inventors: Andrew Lisle WATTS, Hurlstone Park (AU); David PLAYFORD, Mosman Park (AU)

(73) Assignee: ECHOIQ LIMITED, Sydney, New South Wales (AU)

(21) Appl. No.: 18/878,645

(22) PCT Filed: Jun. 30, 2023
(86) PCT No.: PCT/AU2023/050609

§ 371 Date: Dec. 23, 2024

(30) Foreign Application Priority Data
Jul. 1, 2022 (AU) 2022901868

(51) Int. Cl. A61B 8/08; A61B 8/00; G06F 18/214; G06N 3/047; G06N 3/09

(52) U.S. Cl. CPC A61B 8/0883; A61B 5/7267; A61B 5/7275; A61B 8/461; A61B 8/5215; A61B 8/5223; G06F 18/15; G06F 18/214; G06N 3/047; G06N 3/09; G16H 10/60; G16H 20/70; G16H 30/20; G16H 50/20

(57) ABSTRACT

Processing a sparsely populated data source comprising: retrieving data from a sparsely populated data source (a plurality of records) to form a base dataset, each record comprising at least one unpopulated data field corresponding to a medical measurement; dividing the retrieved data into two portions, a training dataset and validation dataset; analyzing the training data set to obtain a trained model and measurement prediction protocols; imputing the predicted measurement values in the records of the training dataset; analyzing the training dataset; validating the disease model, wherein the records of the validation dataset comprise disease data associated with patient data, and determining a validation error; repeating steps to minimize the validation error and computing a prediction of a probable disease state for each patient record in the base dataset.

### FIELD OF THE INVENTION

The present invention relates to artificial intelligence and in particular to systems and methods for artificial intelligence integration to analysis of medical data and records.

The invention has been developed primarily for use in methods and systems for systems and methods for AI-assisted echocardiography for identification of severe aortic stenosis phenotypes and will be described hereinafter with reference to this application. However, it will be appreciated that the invention is not limited to this particular field of use.

### BACKGROUND

Any discussion of the background art throughout the specification should in no way be considered as an admission that such background art is prior art, nor that such background art is widely known or forms part of the common general knowledge in the field in Australia or worldwide.

All references, including any patents or patent applications, cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents forms part of the common general knowledge in the art, in Australia or in any other country.

Phenotype refers to the observable characteristics of an organism as a multifactorial consequence of genetic traits and environmental influences. The organism's phenotype includes its morphological, biochemical, physiological, and behavioural properties. The phenotype, therefore, is the total characteristics displayed by an organism that results from the expression of the genes of an organism as well as the influence of environmental factors experienced by the organism, and random genetic variation.

Echocardiography (or simply “echo”) is a sub-specialty of cardiology that makes use of specialised ultrasound equipment to take diagnostic images of the heart. It is particularly valuable as a first-line diagnostic tool due to its ability to non-invasively assess the internal structure and function of the heart in a cost-effective manner.

A comprehensive echo procedure can measure many features of the heart totalling approximately 150 unique variables, but the full complement of characteristics is rarely measured due to time constraints.

Aggregate echocardiogram data from a wide range of clinics Australia-wide are stored in the National Echocardiogram Database Australia (NEDA). In its current form, NEDA consists of measurements taken from echo procedures and report texts that are an analysis from that procedure for many patients. Currently, the database has echocardiography data relating to more than 1 million patients.

NEDA contains echocardiographic measurement and report data from participating real-world clinical echocardiography laboratories. The measurements are performed as part of standard clinical echocardiography, performed for clinical indications under standard echocardiography imaging protocols. Although there is some variation between laboratories, image acquisition and measurement has been standardised. Guidelines for performing a comprehensive standard transthoracic echocardiogram have been published (see https://www.asecho.org/wp-content/uploads/2018/10/Guidelines-for-Performing-a-Comprehensi ve-Transthoracic-Echocardiographic-Examination-in-Adults.pdf). As is standard in modern echocardiography, the images are stored in DICOM (Digital Imaging and Communications in Medicine) format with the measurements stored in Structured Reporting (SR) format along with the images. At the conclusion of the echocardiogram, the images and SR file are transferred to a Cardiology PACS (Picture Archive and Communications System). From there, the images are viewable with the accompanying measurements, for generation of the echocardiography report. Standard workflow involves a preliminary report by the echocardiographer who performed the study, and the finalised report by a Cardiologist. The final report is that which is sent to the medical record/referring medical practitioner as the definitive interpretation of the echocardiogram procedure. A final echocardiogram report typically contains the measurements that were transferred in the SR file along with the text interpretation of the echocardiogram, and a conclusions section. Recommendations for a standardised transthoracic echocardiogram report can be found at:
https://www.asecho.org/wp-content/uploads/2013/05/Standardized_Echo_Report_Rev1.pdf

For the purpose of this Application, we define “echocardiographic report data” to include all measurement and report information that is contained in the final echocardiogram report. NEDA has developed a proprietary system for capturing all retrospective echocardiographic report data from a participating echocardiography laboratory, allowing for all measured echocardiographic variables and all corresponding interpretive text information to be collated into a single database containing a unique record for each echocardiogram. Each database is then remotely transferred into the Master NEDA Database via a “vendor-agnostic”, automated data extraction process that transfers every measurement for each echocardiogram performed into a standardized NEDA data format (according to the NEDA Data Dictionary). Each individual contributing to NEDA is given a unique identifier along with their demographic profile (date of birth and sex) and all data recorded with their echocardiogram. Using this methodology, NEDA has collected over 1,000,000 echocardiographic reports and subsequently linked this data with the Australian National Deaths Index (NDI) through the data linkage unit at the Australian Institute of Health and Welfare (AIHW), Canberra, Australia. NEDA has obtained multiple ethical approvals from Human Research Ethics Committees (HREC), covering both public and private echocardiography laboratories throughout Australia, as well as the HREC at AIHW for mortality linkage. NEDA is registered by the Australasian Clinical Trials Registry (http://www.anzctr.org.au/ACTRN12617001387314.aspx). All data storage and analyses are maintained and performed on a de-identified basis to protect participant anonymity. This unique resource provides a massive repository of echocardiographic report data that can be used to train artificial intelligence (AI) systems. The NEDA data used to train AI systems is by its nature incomplete. Although there are >150 possible measurements to perform on a transthoracic echocardiogram, it is rarely necessary to perform all the measurements on an individual patient, with typically about ⅓ of measurements performed. Since the individual measurements performed vary on the clinical indication for the echocardiogram and the findings revealed as the echocardiogram is performed, there is no minimum dataset that is present in every echocardiogram. Thus, while NEDA contains a large amount of echocardiographic report data, it may be sparsely populated with certain measurements performed infrequently. FIG. 1 shows a few real examples of different measurements present and missing in echo studies for a small randomly selected group of patients, being a typical example of sparse echo data where each row of the table is a record of the echo measurement available for a single patient.

The NEDA database contains the measurements required to diagnose most cardiac disease that can be identified by echocardiography, with the report data containing additional information obtained by visual inspection of the echocardiographic images. Since each cardiac disease identified by echocardiography has typical features (“phenotype”) that are contained within the measurement and text information, NEDA contains a rich tapestry of disease phenotypes, although each disease phenotype is not labelled (or identified) within the NEDA database. Therefore, NEDA does not include patient phenotype information to identify traits or groups of traits which are held by patients having common diseases.

The workflow for a typical prior art echocardiography study process is depicted in FIG. 2. In summary, a typical workflow consists of the following steps as discussed below.

Images are acquired 101 by a sonographer using a special ultrasound machine.

Specific physical features are measured 103 from the acquired echo images by the sonographer, for example, the diameter of the left ventricle is commonly measured. (Note: many of the measurements will typically be taken during the image acquisition process with the patient present, while others may be measured from the images acquired during the procedure once the patient has left). In this workflow, the sonographer has complete and sole control over whether or not sufficient images of the patient have been acquired or whether additional images are required 104 for a meaningful diagnosis of the patient's actual or suspected condition.

The images and measurements are manually interpreted 105 by the sonographer.

A preliminary report is prepared 107 by the sonographer detailing their interpretation of the study.

The cardiologist reads the preliminary report and inspects the manual analyses 109 and measurements.

The cardiologist creates a final report 111 with their remarks and conclusions from the study.

A key point is that the set of images and measurements required to be taken to ensure that the sonographer or cardiologist has sufficient data to diagnose the patient's condition is comprehensive, meaning an echo study is very time-consuming for the sonographer and prone to error, such as particular data being missed during the scan by a sonographer who may be inexperienced or unfamiliar with the requirements for a particular study. In practice, however, not all possible measurements are taken, only a subset related to a suspected condition or disease are recorded by the sonographer. It is dependent upon the sonographer's skill and experience to know which measurements are important for subsequent analysis and diagnosis.

Accordingly, there is a need for methods of obtaining complete echocardiography datasets for diagnosis of abnormal or disease conditions in patients within time and cost constraints of physical echocardiography procedures. Also, there is a need for methods and systems for determining meaningful data to provide a complete echocardiography record for past echocardiography patients.

Furthermore, there is a need for identifying patients at increased risk from a particular disease such as aortic stenosis. Preferably, the likelihood of a particular disease state in a patient should be identified during the echocardiography investigation. This would enable that the sonographer is advised of additional measurements which should be recorded during the investigation to help the cardiologist to confirm or rule out the identified disease state.

Severe aortic stenosis (AS) is the most common primary valve disease leading to intervention¹. Without timely intervention, AS is associated with progressive myocardial hypertrophy and dysfunction², left atrial dilatation and pulmonary hypertension^{1,3}; the clinical sequalae being heart failure and sudden death. Echocardiography is pivotal to identifying AS and its accompanying adaptive response^{1,3}. However, diagnosis of AS is highly operator dependent⁴ and requires (often scarce⁵) expert interpretation. Additionally, minor errors in measurement of left ventricular outflow tract dimension and velocity time integral are multiplied when calculating the aortic valve area in the continuity equation, a pitfall noted in clinical guidelines¹, which leads to poor diagnosis outcomes.

The transaortic gradient, a robust marker of severe AS³, is heavily influenced by left ventricular systolic function. A lower transaortic gradient in the setting of impaired left ventricular systolic function impacts on interpretation of AS severity. These technical challenges have resulted in calls for improved automation and objective echo interpretation systems⁶. Artificial Intelligence (AI) is a disruptive technology with enormous potential to improve the quality and consistency in echocardiography. Recent work has shown AI has promise in image recognition⁷⁻⁹. However, AI-based interpretation of the comprehensive measurement set performed during echocardiography has not been explored. Consequently, we set out to develop, for the first-time, a robust AI-based system using echocardiographic measurements to interpret the pathophysiology of AS and patient phenotypes which are indicative of AS. Specifically, our aim was to produce a reliable AI system to assist in the clinical diagnosis of severe AS without the need for potentially unreliable left ventricular outflow tract dimension and velocity measurements, and to introduce a robust quality feedback system that could be routinely applied in clinical practice.

### SUMMARY

It is an object of the present invention to overcome or ameliorate at least one or more of the disadvantages of the prior art, or to provide a useful alternative.

The systems and methods disclosed herein provide artificial intelligence (AI) systems for predicting missing measurements from echocardiography data records and providing risk assessments for diseases from incomplete data records with AI populated data.

One embodiment provides a computer program product for performing a method as described herein.

One embodiment provides a non-transitive carrier medium for carrying computer executable code that, when executed on a processor, causes the processor to perform a method as described herein.

One embodiment provides a system configured for performing a method as described herein.

According to a first aspect of the present invention, there is provided a method for processing a sparsely populated data source comprising:
(a) retrieving data from a sparsely populated data source to form a base dataset, the data source comprising a plurality of patient records including patient mortality data, each patient record comprising at least one unpopulated data field corresponding to a medical measurement;
(b) dividing the base dataset into two portions:
   a first portion comprising a training dataset being a defined percentage X % of the base dataset; and
   a second portion comprising a validation dataset being a defined percentage (100%-X %) of the base dataset;
(c) analysing the training data set to jointly model variable relationships using a non-linear function approximation algorithm applied iteratively to the records of the training dataset to obtain a trained model and measurement prediction protocols for populating unpopulated fields in the training data set;
(d) using the measurement prediction protocols, computing prediction values for measurement data for the unpopulated data fields;
(e) imputing the predicted measurement values in the records of the training dataset;
(f) analysing the training dataset on the basis of predefined disease conditions in known patient records of the base dataset to form a phenotype model adapted to associate patient phenotype data to a probability of a disease condition in patient records of the trained data set;
(g) imputing the predicted measurement values in the records of the validation dataset;
(h) validating the phenotype model comprising analysing the validation dataset using the phenotype model, wherein the records of the validation dataset comprise phenotype data associated with patient data, and determining a validation error comprising a probability of correctly predicting a patient phenotype associated with a disease state probability in the records of the validation set;
(i) repeating Steps (c) to (h) to minimise the validation error and computing a prediction of a probable disease state phenotype for each patient record in the base dataset.

Optionally, analysing the training data set is performed using a using a machine learning system.

Optionally, the disease state is aortic stenosis.

Optionally, the sparsely populated data source comprises a plurality of medical records comprising measurement data obtained from a medical study procedure.

Optionally, the medical study procedure comprises an echocardiography procedure.

Optionally, during a measurement procedure, unpopulated measurement data is predicted using the measurement prediction protocols on the basis of data collected by a procedure operator.

Optionally, during a measurement procedure, a patient phenotype is determined by the phenotype model on the basis of data collected by a procedure operator and/or on measurement data predicted using the measurement prediction protocols.

Optionally, the unpopulated measurement data and the patient phenotype is computed in real-time during the measurement procedure.

Optionally, the machine learning system comprises:
a neural network, and during a measurement procedure, measurements obtained by a procedure operator are incorporated into the training data set to form an updated dataset and analysing the updated training data set using the neural network to compute updated measurement prediction protocols and/or an updated phenotype model; and
the measurements obtained during the measurement procedure are analysed using the updated measurement prediction protocols and/or updated phenotype model to predict a probable disease state for a patient undergoing the measurement procedure.

Optionally, as a result of the phenotype prediction associated with a predefined disease state, directing the measurement operator to record relevant measurement data to increase the confidence of the patient phenotype and prediction of an associated disease state.

According to a second aspect of the invention, there is provided an apparatus for conducting a measurement procedure on a patient, the apparatus comprising:
measurement tools relevant to the measurement procedure;
means for recording measurement data from the patient during the measurement procedure; and
means for transmitting the measurement data to an analysis means, said analysis means comprising:
   input means for receiving the measurement data, and phenotype data;
   means for associating the measurement data and phenotype data to determine a patient phenotype associated with one or more disease states;
   measurement prediction protocols for predicting measurement data for unpopulated measurement fields; and/or
   a phenotype model for associating the patient data with a phenotype associated with one or more disease state, thereby to predict a probable disease state for a patient undergoing a measurement procedure; and
   means for alerting the measurement operator of the predicted measurement data and probable disease state and for providing direction to the measurement operator for relevant measurement data to be collected on the basis of the predicted probable disease state.

Optionally, the disease state is aortic stenosis.

Optionally, the apparatus further comprises a display surface adapted to display a notification to the measurement operator comprising the predicted measurement data or a probable disease state.

According to a third aspect of the invention, there is provided a computer implemented method for processing a sparsely populated data source comprising:
(a) retrieving data from a sparsely populated data source to form a base dataset, the data source comprising a plurality of patient records including patient mortality data, each patient record comprising at least one unpopulated data field corresponding to a medical measurement;
(b) dividing the base dataset into two portions:
   a first portion comprising a training dataset being a defined percentage X % of the base dataset; and
   a second portion comprising a validation dataset being a defined percentage (100%-X %) of the base dataset;
(c) analysing the training data set to jointly model variable relationships using a non-linear function approximation algorithm applied iteratively to the records of the training dataset to obtain a trained model and measurement prediction protocols for populating unpopulated fields in the training data set;
(d) using the measurement prediction protocols, computing prediction values for measurement data for the unpopulated data fields;
(e) imputing the predicted measurement values in the records of the training dataset;
(f) analysing the training dataset on the basis of predefined disease conditions in known patient records of the base dataset to form a phenotype model adapted to associate patient phenotype data to a probability of a disease condition in patient records of the trained data set;
(g) imputing the predicted measurement values in the records of the validation dataset;
(h) validating the phenotype model comprising analysing the validation dataset using the phenotype model, wherein the records of the validation dataset comprise phenotype data associated with patient data, and determining a validation error comprising a probability of correctly predicting a patient phenotype associated with a disease state probability in the records of the validation set;
(i) repeating Steps (c) to (h) to minimise the validation error and computing a prediction of a probable disease state phenotype for each patient record in the base dataset.

According to a fourth aspect of the invention, there is provided a computer system comprising:
one or more processors;
one or more memories storing instructions which, when executed by the one or more processors, cause the processors to:
(a) retrieve data from a sparsely populated data source to form a base dataset, the data source comprising a plurality of patient records including patient mortality data, each patient record comprising at least one unpopulated data field corresponding to a medical measurement;
(b) divide the base dataset into two portions:
   a first portion comprising a training dataset being a defined percentage X % of the base dataset; and
   a second portion comprising a validation dataset being a defined percentage (100%-X %) of the base dataset;
(c) analyse the training data set to jointly model variable relationships using a non-linear function approximation algorithm applied iteratively to the records of the training dataset to obtain a trained model and measurement prediction protocols for populating unpopulated fields in the training data set;
(d) use the measurement prediction protocols, computing prediction values for measurement data for the unpopulated data fields;
(e) impute the predicted measurement values in the records of the training dataset;
(f) analyse the training dataset on the basis of predefined disease conditions in known patient records of the base dataset to form a phenotype model adapted to associate patient phenotype data to a probability of a disease condition in patient records of the trained data set;
(g) impute the predicted measurement values in the records of the validation dataset;
(h) validate the phenotype model comprising analysing the validation dataset using the phenotype model, wherein the records of the validation dataset comprise phenotype data associated with patient data, and determining a validation error comprising a probability of correctly predicting a patient phenotype associated with a disease state probability in the records of the validation set; and
(i) repeat Steps (c) to (h) to minimise the validation error and computing a prediction of a probable disease state phenotype for each patient record in the base dataset.

According to a fifth aspect of the invention, there is provided a computer program product having a computer readable medium having a computer program recorded therein for processing a sparsely populated data source, said computer program product comprising:
(a) computer program code means for retrieving data from a sparsely populated data source to form a base dataset, the data source comprising a plurality of patient records including patient mortality data, each patient record comprising at least one unpopulated data field corresponding to a medical measurement;
(b) computer program code means for dividing the base dataset into two portions:
   a first portion comprising a training dataset being a defined percentage X % of the base dataset; and
   a second portion comprising a validation dataset being a defined percentage (100%-X %) of the base dataset;
(c) computer program code means for analysing the training data set using a non-linear function approximation algorithm applied iteratively over the records of the training data set to obtain a trained data set and measurement prediction protocols for populating unpopulated field in the training data set;
(d) computer program code means for using the measurement prediction protocols, computing prediction values for measurement data for the unpopulated data fields;
(e) computer program code means for imputing the predicted measurement values in the records of the training dataset;
(f) computer program code means for analysing the training dataset on the basis of predefined disease conditions in known patient records of the base data set to form a phenotype model adapted to associate patient phenotype data to a probability of a disease condition in patient records of the trained data set;
(g) computer program code means for imputing the predicted measurement values in the records of the validation dataset;
(h) computer program code means for validating the phenotype model comprising analysing the validation dataset using the phenotype model, wherein the records of the validation dataset comprise phenotype data associated with patient data, and determining a validation error comprising a probability of correctly predicting a patient phenotype associated with a disease state probability in the records of the validation set;
(i) computer program code means for computing a prediction of a probable disease state phenotype for each patient record in the base dataset.

Optionally, the disease state is aortic stenosis.

### BRIEF DESCRIPTION OF THE DRAWINGS

Notwithstanding any other forms which may fall within the scope of the present invention, preferred embodiments of the invention will now be described, by way of example only, with reference to the accompanying drawings in which:

FIG. 1 shows an example of patient records in a sparsely populated dataset;

FIG. 2 shows a typical workflow procedure for existing echocardiography analysis methods;

FIG. 3 shows a typical workflow of an AI Assisted Reporting mode of echocardiography analysis according to an embodiment of the invention as disclosed herein;

FIG. 4 shows a typical workflow of an AI-In-The-Loop mode of echocardiography analysis according to an embodiment of the invention as disclosed herein;

FIG. 5 shows a schematic representation of the major components of an AI-architecture system adapted for AI-assisted echocardiography as disclosed herein;

FIG. 6 shows a schematic representation of a network accessible application implementation of the AI-assisted echocardiography methods and systems as disclosed herein;

FIG. 7 shows a computing device on which the various embodiments described herein may be implemented in accordance with an embodiment of the present invention;

FIG. 8 shows graphical results of an embodiment of the AI-assisted echocardiography methods and systems as disclosed herein for prediction of severe Aortic Stenosis (AS) in the NEDA records for the general population;

FIGS. 9A, 9B, 9C and 9D show graphical results of a the prediction accuracy of an embodiment of the AI-assisted echocardiography methods and systems as disclosed herein for prediction of severe Aortic Stenosis (AS) in the NEDA records for subsets of the general population, respectively for ejection fraction value (EF) of <=50%, EF<=40%, EF<=35%, and EF<=30%;

FIG. 10 shows a schematic representation of the overall architecture of the AI model and the training process;

FIG. 11 shows the error distribution plots when the trained AI model was applied to the test subset;

FIG. 12 shows a Table 4 of a comparison of variables for patients with and without severe AS;

FIG. 13 is a schematic depiction of the flow 900 of analyses performed on data derived from the study cohort of Example 2;

FIG. 14 depicts AUC curves of AI-augmented diagnosis of severe AS compared with that derived from the continuity equation from the study in Example 2; and

FIG. 15 depicts actuarial 5-year mean survival for the AI diagnosis of non-severe AS from the study in Example 2.

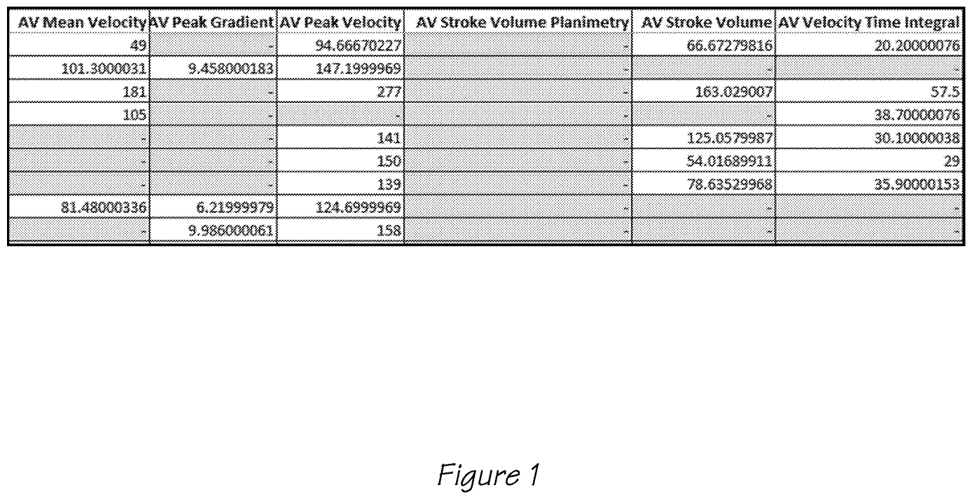

### DEFINITIONS

The following definitions are provided as general definitions and should in no way limit the scope of the present invention to those terms alone, but are put forth for a better understanding of the following description.

Unless defined otherwise, all technical and scientific terms used herein have the same meaning as commonly understood by those of ordinary skill in the art to which the invention belongs. It will be further understood that terms used herein should be interpreted as having a meaning that is consistent with their meaning in the context of this specification and the relevant art and will not be interpreted in an idealized or overly formal sense unless expressly so defined herein. For the purposes of the present invention, additional terms are defined below. Furthermore, all definitions, as defined and used herein, should be understood to control over dictionary definitions, definitions in documents incorporated by reference, and/or ordinary meanings of the defined terms unless there is doubt as to the meaning of a particular term, in which case the common dictionary definition and/or common usage of the term will prevail.

For the purposes of the present invention, the following terms are defined below.

The articles “a” and “an” are used herein to refer to one or to more than one (i.e., to at least one) of the grammatical object of the article. By way of example, “an element” refers to one element or more than one element.

The term “about” is used herein to refer to quantities that vary by as much as 30%, preferably by as much as 20%, and more preferably by as much as 10% to a reference quantity. The use of the word ‘about’ to qualify a number is merely an express indication that the number is not to be construed as a precise value.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

Any one of the terms: “including” or “which includes” or “that includes” as used herein is also an open term that also means including at least the elements/features that follow the term, but not excluding others. Thus, “including” is synonymous with and means “comprising”.

In the claims, as well as in the summary above and the description below, all transitional phrases such as “comprising”, “including”, “carrying”, “having”, “containing”, “involving”, “holding”, “composed of”, and the like are to be understood to be open-ended, i.e., to mean “including but not limited to”. Only the transitional phrases “consisting of” and “consisting essentially of” alone shall be closed or semi-closed transitional phrases, respectively.

The term, “real-time”, for example “displaying real-time data”, refers to the display of the data without intentional delay, given the processing limitations of the system and the time required to accurately measure the data.

The term “near-real-time”, for example “obtaining real-time or near-real-time data” refers to the obtaining of data either without intentional delay (“real-time”) or as close to real-time as practically possible (i.e., with a small, but minimal, amount of delay whether intentional or not within the constraints and processing limitations of the of the system for obtaining and recording or transmitting the data.

Although any methods and materials similar or equivalent to those described herein can be used in the practice or testing of the present invention, preferred methods and materials are described. It will be appreciated that the methods, apparatus and systems described herein may be implemented in a variety of ways and for a variety of purposes. The description here is by way of example only.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The phrase “and/or”, as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of”, or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either”, “one of”, “only one of”, or “exactly one of”. “Consisting essentially of”, when used in the claims, shall have its ordinary meaning as used in the field of patent law.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B”, or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

For the purpose of this specification, where method steps are described in sequence, the sequence does not necessarily mean that the steps are to be carried out in chronological order in that sequence, unless there is no other logical manner of interpreting the sequence.

In addition, where features or aspects of the invention are described in terms of Markush groups, those skilled in the art will recognise that the invention is also thereby described in terms of any individual member or subgroup of members of the Markush group.

### DETAILED DESCRIPTION

It should be noted in the following description that like or the same reference numerals in different embodiments denote the same or similar features.

As discussed above, the systems and methods disclosed herein provide artificial intelligence (AI) systems and methods for predicting missing measurements from echocardiography data records as well as identifying patient phenotypes which are indicative of aortic stenosis (AS) or similar disease states, and providing risk assessments for diseases from incomplete data records with AI populated data.

Discussed below (particularly in Paragraphs to of this document) is an implementation of an AI method in which a machine learning system comprising a supervised neural network (in the form of a Mixture Density Network) is trained to output a probabilistic imputation of missing data and secondary classification algorithm applies clinically determined thresholds to the imputed outputs in order to predict the presence of a disease phenotype. The neural network is internally validated by randomly holding out training data and minimizing the imputation error. Validation of the classification algorithm is discussed in Paragraphs to but can be summarized as follows:

The classification algorithm is validated by optimizing performance metrics on the validation dataset. The performance of the classification algorithm in predicting the AS phenotype demonstrated an AUROC of 0.9696 (see in particular, Paragraph [0218]). The AI diagnosis remained highly predictive of death after adjustment for age and gender (see in particular, Paragraph [0220]).

External validation has been carried out in the form of several independent clinical trials conducted in Australia and the United States which have consistently identified additional patients with aortic stenosis who fell outside diagnostic guidelines but showed significant risk of dying from the disease. In one particular study, historical echocardiograph records from 9189 patients were analysed using the AI systems and methods disclosed above. In contrast to the diagnosis at the time of the echo examination by medical professionals, the AI system described herein found a 72% increase in the instance of severe Aortic Stenosis. It is well known that AS often goes undiagnosed in female patients, possibly due to gender-based differences in the heart muscles and this study also found that women were 66% more likely to have been misdiagnosed with human-only diagnosis than men. Systems and methods such as the AI system described above which look as many multiples more data points in an entirely objective manner have significant potential in supporting improved patient outcomes but correctly identifying disease to allow the patient to receive timely treatment where appropriate. It is clear that AI systems and methods such as those disclosed herein can be invaluable tools to supplement the expertise of the medical professional.

A de-identified copy of the NEDA database containing the full range of measurements, including mortality data is used for creating an AI model as discussed below. Using a random 70% subset of patients, a modified Mixture Density Network¹⁰ is trained to serve as a multiple-imputation model. The model is trained with missing data by augmenting the model with Boolean inputs signalling whether a measurement was present or not to predict severe AS.

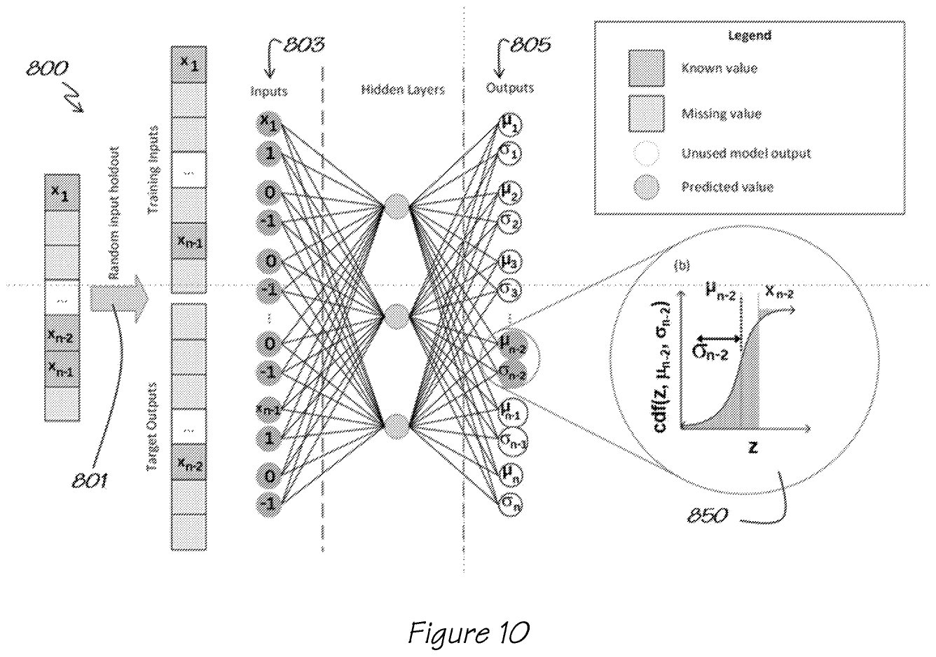

In particular, example data entries from the NEDA data source had measurements randomly held out from the training inputs and used as target outputs for regression. Backpropagation is only applied to model outputs with a target output present. In this way, the training examples approximately resemble typical sets of measurements encountered in echo without requiring complete sets of measurements for the training process. The resulting model is designed to be general-purpose and can perform inference using arbitrary sets of available measurements. The atypical backpropagation procedure utilised herein can be viewed as training a family of models with shared weights (this has some similarities to techniques previously applied to restricted Boltzmann machines¹⁵). The input holdout process has similarities to the common technique known as dropout (although in this case rather than using the existing method of discarding the “dropped out” values they are used to build the sentinel vector and the target outputs) and is seen to have a secondary effect of regularizing the model, thereby encouraging the learning of more generalizable patterns¹¹. The continuous rank probability score (CRPS) is chosen as the loss function since it has a closed-form solution for a mixture-of-Gaussians¹² and encourages convergence to sharp and well-calibrated predictions¹³. Intuitively, the CRPS loss function penalises the model for predicting an incorrect expected value while also penalising over or under confident predicted distributions. FIG. 10 depicts the overall architecture 800 of the model and the training process. The overall training process consisting of random input holdout 801 followed by backpropagation from the target outputs. Inputs 803 [x₁ . . . xₙ] are echo measurements with missing values, outputs 805 (μᵢ, σᵢ); where i∈{1, . . . , n} denote Gaussian prediction densities with mean μᵢ and standard deviation σᵢ (but the general approach may be applied to any choice of density function with closed-form solution for CRPS including mixture of Gaussians). Magnified section 850 depicts the cumulative density function (CDF) of the prediction xₙ₋₂~ [symbol] (μₙ₋₂, σₙ₋₂) plotted for possible values of the measurement z and compared with the target value. CRPS is calculated by integrating the squared height of the shaded area over all values of z.

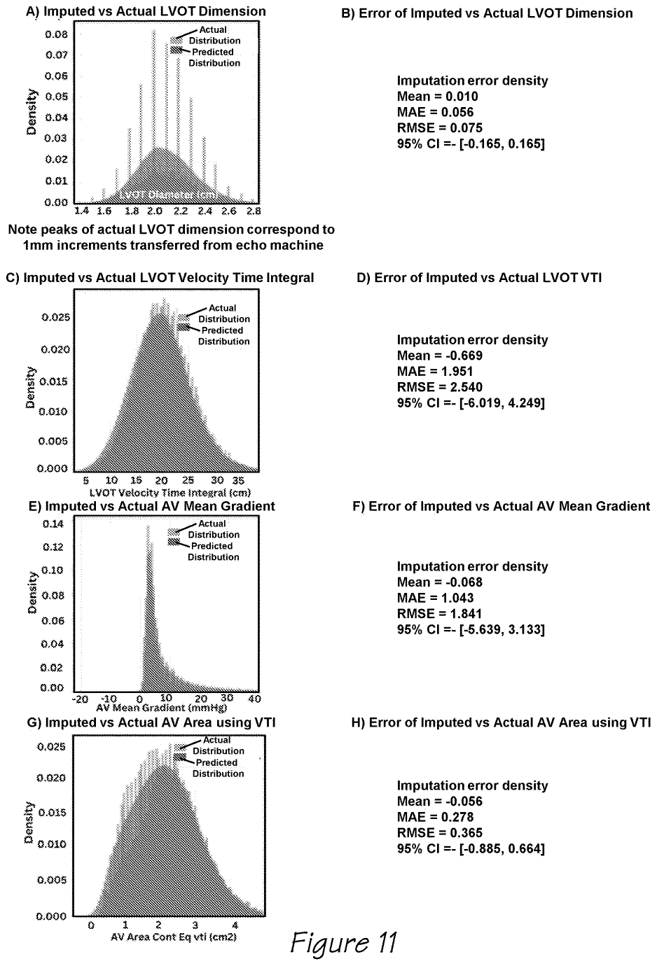

The model was tested by applying it to the remaining 30% test subset of patients not used for training. As an initial diagnostic, selected groups of related measurements were withheld and the AI predictions were evaluated against the known measured values. These results indicated the predicted measurements had minimal bias and surprisingly low error bounds considering the heterogeneous nature of the data and the fact that key information (i.e., left ventricular outflow tract data) had been removed from the studies. FIG. 11 shows the error distribution plots when the trained model was applied to the test subset. Demonstration of the error plots for prediction of measurements in the 30% test set.

In FIG. 11, the results show minimal bias and low error rates considering the heterogeneous nature of the data and the fact that key information (i.e., LVOT data) has been removed from the studies. The panels on the left demonstrate the imputed vs actual measurements overlaid. The panels on the right demonstrate the imputation error (imputed vs actual measurement), calculated after predicting while holding out each measurement plus any directly dependent variables. The mean errors (95% confidence interval) were as follows: LVOT dimension=0.010 cm (−0.165 to 0.165), LVOT velocity time integral=−0.669 cm (−6.019 to 4.249), Mean transaortic valve gradient=0.068 mmHg (−5.639 to 3.133), Aortic Valve Area=−0.056 cm² (−0.885 to 0.664), p=ns for each imputed vs actual measurement.

The AI predictions for the continuity-derived aortic valve area were then evaluated in the clinical context of classifying severe AS. Initially, the test set data was filtered to only consider studies with a known aortic valve area calculated using the continuity equation and used this to label the studies as “severe AS” or “not severe AS”. Then, all left ventricular outflow tract measurements (velocity, gradient and diameter), the aortic valve area, and the aortic root dimensions were left out from this test set and the model was used to predict a distribution of likely values for the aortic valve area. A predicted probability of severe AS was derived by evaluating the cumulative density function of the predictions to calculate p(AVA<1 cm²). ROC analysis was then performed to quantify classifier performance by comparing the AI predicted probabilities with the original classification label using the standard, continuity-derived AVA. It will be readily appreciated by the skilled addressee that this method lends itself to the application of this type of model to existing workflows, processes and guidelines.

### Severe Aortic Stenosis

AS is evaluated from echocardiograph data using measurements such as, for example, the peak aortic jet velocity, aortic mean gradient, and the aortic valve area defined by the Continuity Equation14 (CE):

$$AVA ( {cm}^{2} ) = \frac{{CSA}_{LVOT} \times {VTI}_{LVOT}}{{VTI}_{AV}} \qquad ( CE )$$

$$where :$$

$${CSA}_{LVOT} is the cross sectional area of the left ventricular outflow tract ;$$

$$LVOT = π {D}^{2} / 4 and D = LVOT dimension ( cm ) ;$$

$${VTI}_{LVOT} is the velocity time integral of the LVOT velocity trace ; and$$

$${VTI}_{AV} is the velocity time integral of the aortic value velocity trace .$$

Severe AS is defined as an AVA <1.0 cm² (highest measured VTI_{AV} and the mean VTI_{LVOT}).

### Characteristics of Patients Identified as Severe AS by AI

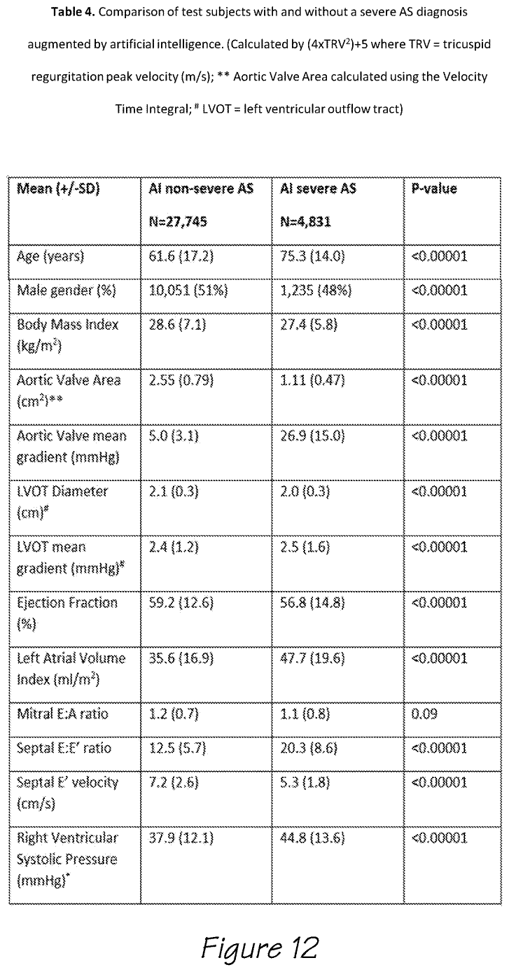

Patients identified by the AI system as having severe AS had the expected demographic and clinical characteristics (see Table 4 in FIG. 12), with increased aortic valve gradients, impaired left ventricular diastolic function, and increased indexed left ventricular mass, indexed left atrial volume and right ventricular systolic pressure. Patients diagnosed with severe AS by the AI system but not by the continuity equation (CE) had similar characteristics to those with severe AS by continuity, except for a lower transaortic gradient and stroke volume index, consistent with the AI's interpretation of typical cardiac structural changes in response to aortic stenosis.

Characteristics of those patients diagnosed with AS are used to identify the phenotypic characteristics associated with AS to apply such phenotypic characteristics to new patient data. Mortality data from the NEDA database is also considered in conjunction with the Patient characteristics with confirmed AS to provide an improved phenotype characteristic set so as to provide a more accurate prediction of an AS diagnosis.

### Application to a Limited Data Set

To determine which echocardiographic parameters most influenced the AI in making its prediction, measurements were chosen which classically represent a pressure-loaded left ventricle in severe AS. These were then used to create a limited test set which contained only the following variables as model inputs (where known): gender, height, weight, basal 2D dimensions (ventricular septal and posterior wall diastolic thickness, left ventricular internal dimension in systole and diastole), left ventricular ejection fraction measured using the Simpson's Biplane method, ascending aortic dimension, atrial measurements (left atrial area in 4- and 2-chamber, 4-chamber left atrial length, right atrial area), mitral inflow pulsed wave Doppler data (E velocity, A velocity, E wave pressure half-time, mitral inflow velocity time integral), left ventricular diastolic basal tissue Doppler velocities (E′ septal velocity, E′ lateral velocity), transaortic velocities (aortic peak velocity, aortic velocity time integral), and pulmonary valve peak velocity.

### (a) Complete Set of Measured Variables

The full set of variables used for training the artificial intelligence in the “comprehensive” exam were as discussed below.
General—Body Height, Body Weight, Body Mass Index, Body Surface Area, Gender, Age, Heart Rate
2-Dimensional Measurements IVS Diastolic Thickness, LVPW Diastolic Thickness, LA Area, LA Length 4C, LA Systolic Diameter LX, LA Volume, LA Volume Index, LV Diastolic Area 2C, LV Diastolic Area 4C, LV Diastolic Area PSAX, LV Diastolic Diameter PLAX, LV Systolic Diameter MM, LV Systolic Diameter PLAX, LV Diastolic Length 2C, LV Diastolic Length 4C, LV Diastolic Volume 2C, LV Diastolic Volume 2D Teich, LV Diastolic Volume 4C, LV Diastolic Volume BP, LV Ejection Fraction 2C, LV Ejection Fraction 4C, LV Ejection Fraction BP, LV Stroke Volume 2C AL, LV Stroke Volume 2D Teich, LV Stroke Volume 4C AL, LV Stroke Volume MOD 2C, LV Stroke Volume MOD 4C, LV Stroke Volume MOD BP, LV Stroke Volume SIM, LV Systolic Area 2C, LV Systolic Area 4C, LV Systolic Diameter 4C, LV Systolic Length 2C, LV Systolic Length 4C, LV Systolic Volume 2C AL, LV Systolic Volume 2D Teich, LV Systolic Volume 4C AL, LV Systolic Volume MOD 2C, LV Systolic Volume MOD 4C, LV Systolic Volume MOD BP, Ejection Fraction (any method), Aorta at Sinotubular Diameter, Aorta at Sinuses Diameter, Ascending Aorta Diameter, Distal Transverse Aorta Diameter, IVC Diameter Expiration, IVC Diameter Inspiration, LV Epi Diastolic Area PSAX, LV Mass AL, LV Mass AL Index, RA Area, Right Atrial Volume, Right Atrial Volume Index.
Doppler Measurements—MV Mean Gradient, MV Mean Velocity, MV Peak Gradient, MV Peak Velocity, MV Pressure Half Time, MV Velocity Time Integral, MV Area PHT, MV Deceleration Slope, MV Deceleration Time, Mitral A wave Velocity, Mitral E wave Velocity, Mitral E to A Ratio, MV E′ Septal Velocity, LV E′ Lateral Velocity, LV A′ Velocity, Mitral E to E Prime Septal Ratio, Mitral E to E Prime Lateral Ratio, Mitral mean E to E′ Ratio, AR Peak Velocity, AR Pressure Half Time, AV Mean Gradient, AV Mean Velocity, AV Peak Gradient, AV Peak Velocity, AV Velocity Time Integral, Pulm Vein Atrial Duration, Pulm Vein Atrial Reversal Velocity, Pulmonary Vein A Velocity, Pulmonary Vein Diastolic Velocity, Pulmonary Vein S/D Ratio, Pulmonary Vein Systolic Velocity, TR Peak Gradient, TR Peak Velocity, Right Ventricular Systolic Pressure, PV Mean Gradient, PV Mean Velocity, PV Peak Gradient, PV Peak Velocity, PV Velocity Time Integral, RVOT Mean Gradient, RVOT Mean Velocity, RVOT Peak Gradient, RVOT Peak Velocity, RVOT Velocity Time Integral

### (b) Reduced/Limited Set of Measured Variables

The set of variables included in the “limited” exam were as discussed below.
General—Gender, Body Height, Body Weight, Body Mass Index, Body Surface Area.
2-Dimensional Measurements—Ejection Fraction (by any method), LV Systolic Diameter Base PLAX, LV Diastolic Diameter base PLAX, LV Systolic Diameter PLAX, LVPW Diastolic Thickness, IVS Diastolic Thickness, LA Area, RA Area, Aorta at Sinotubular Diameter, Aorta at Sinuses Diameter, Ascending Aorta Diameter.
Doppler Measurements—AV Mean Gradient, AV Mean Velocity, AV Peak Gradient, AV Peak Velocity, AV Velocity Time Integral, Mitral E′ velocity septum, Mitral E′ velocity lateral, Mitral E to E Prime Ratio Septal, Mitral E to LV E Prime Ratio Lateral, Mitral E to MV E Prime Ratio Mean, MV Mean Gradient, MV Mean Velocity, MV Peak Gradient, MV Peak Velocity, MV Pressure Half Time, MV Velocity Time Integral, Mitral A Point Velocity, Mitral E Point Velocity, Mitral E to A Ratio, PV Peak Gradient, PV Peak Velocity.

This limited data set takes approximately 10 minutes to acquire when performing a transthoracic echocardiogram. All other variables were held out from the test set, including left ventricular outflow tract measurements, and the AI was evaluated using the same methodology described above. This is defined as the “limited echocardiography” system, and the output classification was a probability of severe AS, as described above.

### Imputation

Echocardiographic studies are time-consuming, and so for reasons of efficiency an echo study will only focus on measurements deemed relevant by the cardiologist. This means that the NEDA database contains an incomplete set of measurements for each patient, which presents as an obstacle to the use of many techniques from the fields of statistics and machine learning.

A naïve solution to this problem is “complete case analysis”, in which a subset of measurements are selected and any patient data with an incomplete set of measurements is discarded. This approach is flawed for two reasons:
it results in large amounts of useful information being discarded; and
it introduces sampling bias—the fact that a subpopulation of patients have a certain set of measurements is likely to be associated with a specific family of diseases. In statistical terms, the measurements are Missing Not At Random (MNAR).

A better alternative is to “fill in the blanks” or, in statistical terms, to impute the missing values.

### Phenotype Identification

The AI engine described above is also configured to determine the typical phenotypes for patients which have aortic stenosis (AS) or similar disease states by identifying phenotypes of increased risk that have characteristics that are similar to those characteristics which are observed with aortic stenosis. Specifically, the AI engine is intended for application using echocardiographic and mortality data to predict the phenotype of risk that may be found in aortic stenosis and other diseases with similar characteristics.

The general characteristics of AS are as follows. There may be an abnormality in the velocity across the aortic valve in systole, associated with high flow rates, normal flow rates, or low flow rates. There is a narrowing of the aortic valve associated with a decrease in the aortic valve area, aortic valve calcification, bicuspid aortic valve, unicuspid aortic valve, or an abnormal aortic valve orifice for other reasons. There is either increased, normal, or low flow across the left ventricular outflow tract. The left ventricular dimension may be normal, increased, or small.

The left ventricular systolic function may be normal, hyperdynamic or impaired, measured using the left ventricular ejection fraction, fractional shortening, left ventricular systolic and diastolic volume, or left-ventricular systolic and diastolic dimension.

The left ventricular wall thickness may be normal, increased, or decreased, and associated with a change in left-ventricular mass (may be normal, increased, or decreased). Left-ventricular diastolic function may be normal or abnormal and associated with the following measures: Mitral E wave velocity, mitral A wave velocity, mitral E/A ratio, septal e′ velocity, lateral e′ velocity, septal E:e′ ratio, lateral E:e′ ratio, global longitudinal strain, left atrial area, left atrial dimension, left atrial width, left atrial volume, left atrial volume index, right ventricular systolic pressure, tricuspid regurgitation velocity, right atrial pressure, and right atrial area.

In the setting of severe aortic stenosis, there is a typical phenotype of abnormalities. The AI engine described above is intended to identify this phenotype, noting that this phenotype may also be found in other similar or related diseases such as amyloid cardiomyopathy, hypertensive heart disease, infiltrative cardiomyopathies, restrictive cardiomyopathies, left ventricular outflow tract obstruction, left-ventricular outflow tract membranes, supravalvular aortic stenosis, and impaired valvular haemodynamics post aortic valve replacement. The artificial intelligence identifies the changes that are typical of aortic stenosis that may also be found in anyone or a combination of individual diseases within this set of diseases.

The typical phenotype is as follows, but as described above there many variations. In severe aortic stenosis, there is typically an elevation of the transvalvular aortic gradient, associated with a small aortic valve area, and normal left ventricular outflow tract velocities. There is a normal left ventricular cavity size and left ventricular chamber volume, associated with a normal Left ventricular ejection fraction, and normal stroke volume. There is an increase in left ventricular wall thickness and left-ventricular mass. There are decreased left ventricular myocardial relaxation velocities (septal and lateral e′ velocities) along with elevations in the mitral inflow E wave velocity and normal (pseudo-normal) E/A ratio. A-wave velocities may be decreased. There may be signs of increased left-ventricular filling pressure with an increase in the E:e′ ratio, both septal and lateral. Left atrial volume is increased, associated with increased left atrial pressure, elevated tricuspid regurgitation velocity, and increased right ventricular systolic pressure (signs of pulmonary hypertension).

An important variation of the aortic stenosis phenotype is in the setting of impaired systolic function. Again, there any variations but a typical scenario is described as follows. In severe low flow low gradient aortic stenosis there is increased (or normal) transaortic velocities and transvalvular aortic gradients, associated with a small aortic valve area and low left-ventricular outflow tract velocities. Left ventricular cavity size may be normal or increased, associated with an impaired Left ventricular ejection fraction, and decreased stroke volume. There is an increase in left-ventricular wall thickness and left-ventricular mass. There are decreased left ventricular myocardial relaxation velocities (septal and lateral e′ velocities) along with elevations in the mitral inflow E wave velocity and normal (pseudo-normal) E/A ratio. A-wave velocities may be decreased. There may be signs of increased left-ventricular filling pressure with an increase in the E:e′ ratio, both septal and lateral. Left atrial volume is increased, associated with increased left atrial pressure, elevated tricuspid regurgitation velocity, and increased right ventricular systolic pressure (signs of pulmonary hypertension).

### AI-Assisted Reporting

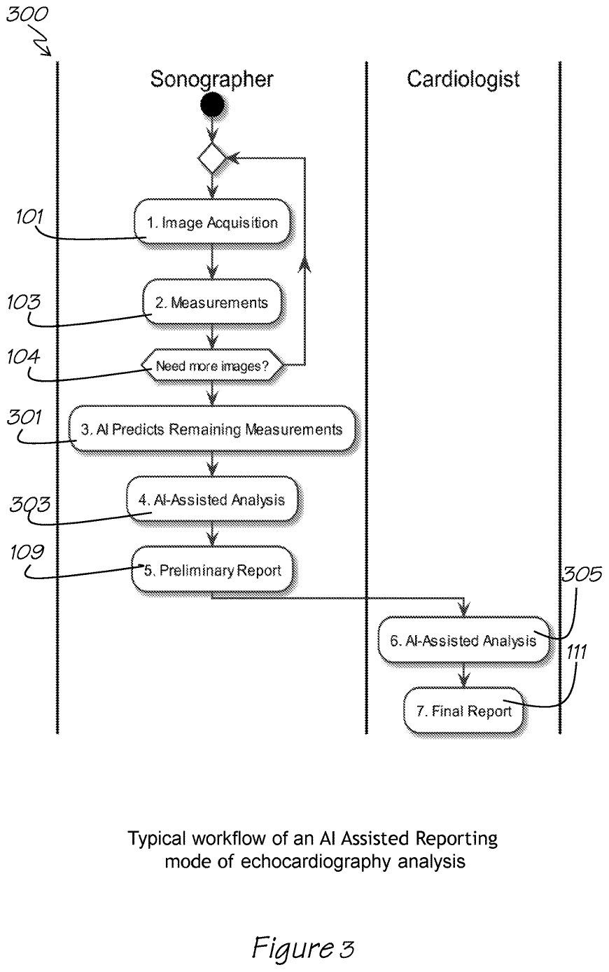

In an embodiment disclosed herein, there is provided an AI-assisted echocardiography reporting aid. In the present embodiment, an echocardiography examination of a patient is carried out as usual and the AI-model is then used to augment the study with a set of predictions and determination of patient phenotype characteristics. This process 300 is shown schematically in FIG. 3.

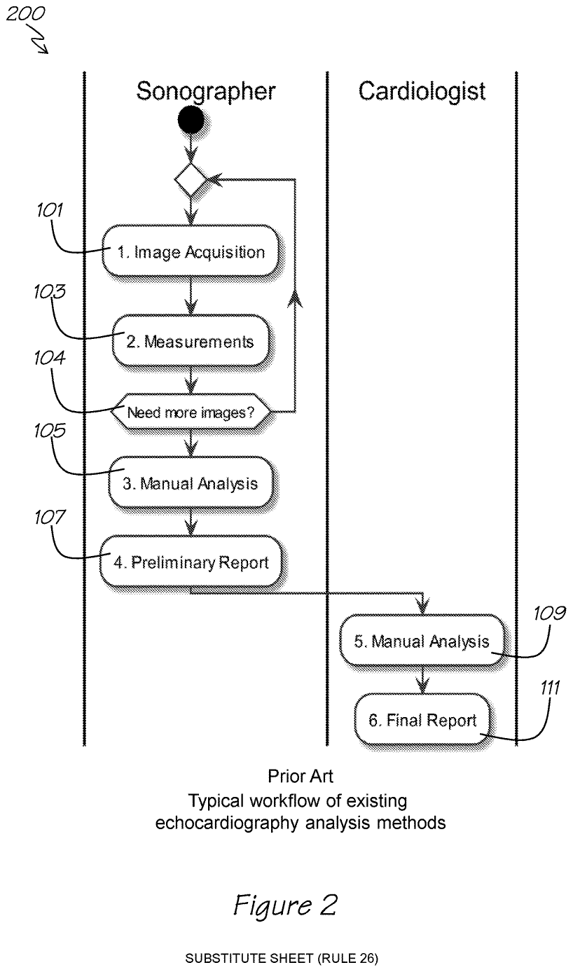

The major difference between the prior art process 200 of FIG. 2 and the AI-assisted reporting procedure 300 presented in FIG. 3 is the addition of AI-model predictions 301 to impute any missing measurement data into the patient's measurement record for the scan, which in turn enables step 4—“AI-Assisted Analysis” 303. The AI-Assisted Analysis 303 uses actual measurements acquired by the sonographer at step 1 101 and may, optionally, also include predictions by the AI-model of missing measurement parameters from step 3 301, to provide computed estimates of the patient's phenotype characteristics and risk of a possibly associated disease state to the healthcare professional (e.g., the sonographer or cardiologist) analysing the study. The sonographer then prepares the preliminary report 109 including measurement data recorded during the scan and, if utilised, measurement data imputed into the scan record by the AI system, and the report is forwarded to the cardiologist for further analysis. In addition to the AI-assisted analysis undertaken by the sonographer in step 4 303, the cardiologist may also, optionally, use an AI system (either the same as that used by the sonographer or a different AI model) to analyse 305 the scanned and/or imputed measurement data in the context of determining the patient's general state of health or their risk of having of possible progressing to a diseased state.

Either the sonographer or the cardiologist can refer to the phenotype characteristics determined by the AI-model and compare it to phenotypes having a known association with AS or similar disease states.

The key efficiency gain from process 300 of prior art process 200 is in the use of AI-assisted analysis techniques as discussed herein to reduce of the time spent by both the sonographer and the cardiologist in manually checking the measurements for the presence or absence of abnormalities in the patient's scan results which would lead to a particular diagnosis of the patient's state of health. Process 300 also has the significant advantage of optionally being configured to identify other potential patient abnormalities based on predicted data for missing measurement data imputed into the patient's scan record, which helps the healthcare professional to pick up on subtle or uncommon conditions which may otherwise be missed by a less experienced sonographer or cardiologist. In these cases, a disease condition may otherwise progress untreated, leading it to only be detected later when more extreme symptoms have manifested. This scenario of the prior art process where subtle or less common disease indicators are missed by the healthcare providers leads to worse patient outcomes and typically also increased costs to healthcare providers.

### AI in the Loop

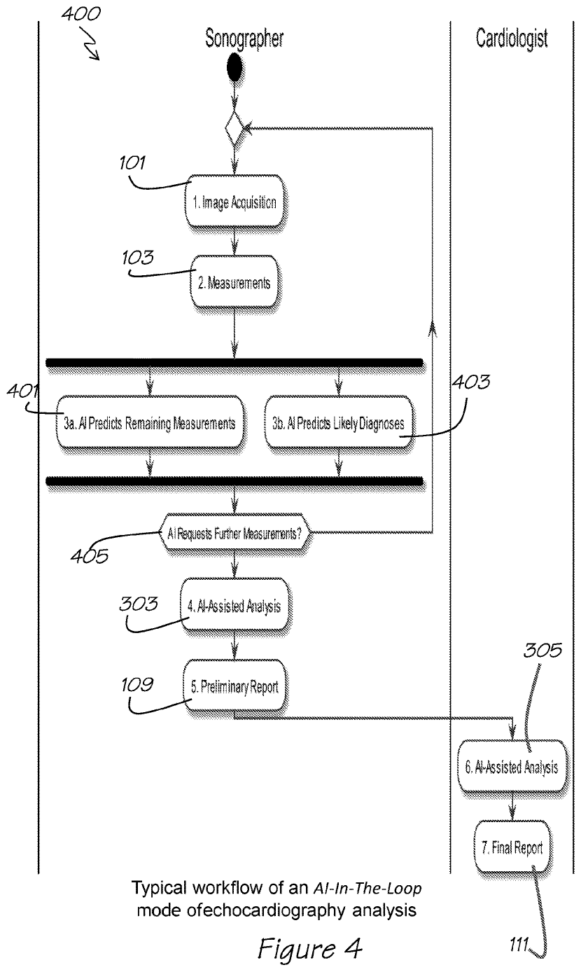

A more advanced application of the AI models disclosed herein is to integrate directly with the measurement process performed by the sonographer whilst taking the measurements of the patient during a scan-“AI-In-The-Loop”. This configuration of the AI model is adapted to provide real-time predictions of various echocardiography measurements to the sonographer whilst they are performing the scan on the patient. FIG. 4 provides a schematic representation of the workflow for the AI-In-The-Loop configuration.

The major benefit of this approach is that certain measurements may not be required to be taken by the sonographer as they can be predicted 401 by the system in real-time while the scan is in progress. The system may also update a prediction of one or more possible diagnoses 403 of condition(s) the patient may have on the basis of the measurements acquired by the sonographer in conjunction with the predicted measurements 401. If any AI-predicted measurements have a high enough “confidence” output from the system, they can be used as-is, saving time. Alternatively, if the sonographer is not satisfied of the confidence level of the predicted measurement, then they could make the particular measurement manually whilst the patient is present. The system may also optionally request 405 particular measurements to be acquired by the sonographer on the basis of the predicted diagnoses 403, for example to manually acquire a particular measurement that may be useful in confirming or ruling out a particular predicted diagnosis made on the basis of existing acquired and predicted measurements.

The method 400 proposed in FIG. 4 is a particularly compelling proposition, particularly in light of the fact that new measurement techniques are continually appearing in the literature and echo specialists (particularly sonographers) are increasingly required to prioritise which measurements to perform in the limited time available for a study.

A second benefit of the method 400 is that in the case of a subtle abnormality in patient's heart, the system is also configured to suggest further measurements to the sonographer and the data may be acquired on-the-spot whilst the patient is present. This is a significant improvement over the method 300 of FIG. 3 which is only able to flag abnormalities to the healthcare professional after the patient has left the clinic, meaning the patient may be required to return for an expensive and time consuming second physical examination.

Furthermore, phenotype characterisation of the patient during the echocardiography examination has particular advantages since if identification of a phenotype associated with AS or similar disease is evaluated, the AI-model can inform the sonographer during the echocardiography examination of a possible positive association and, in turn can direct the sonographer to record one or more additional measurements of the patient whilst the patient is available and the additional measurements may be collected on the spot with needing to recall the patient after the initial examination to conduct further investigations and record additional measurements to either confirm or rule out a possible disease state.

### System Architecture

Recent practices in the European Patent Office have required a highly detailed description of the actual processes used for training and implementing AI systems. Please fill out the following description with as much specific detail as possible.

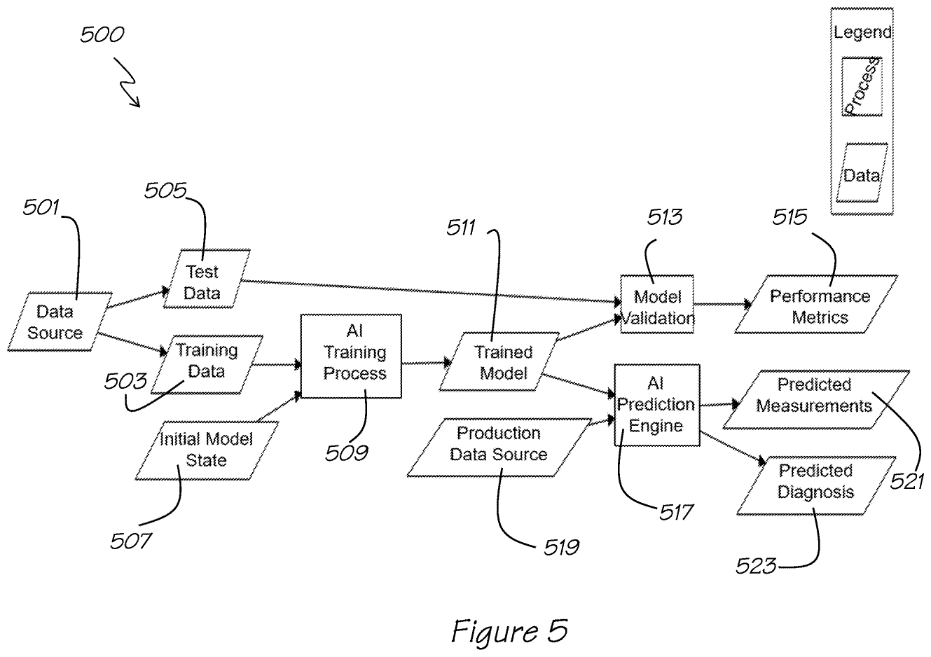

FIG. 5 depicts the major components of an AI-architecture 500 adapted for AI-assisted echocardiography as disclosed herein and the interconnections and interactions between the component parts of system 500. Implementation details of the particular components of system 500 will typically vary by application and are subject to change and so are considered largely out-of-scope for this document.

System 500 is particularly adapted to connect with a database 501 of medical records which may be any form of structured medical data, which could include data derived from medical images, measurements taken during a procedure or even the output of a Natural Language Processing (NLP) algorithm from reading existing medical reports. In the embodiments disclosed herein, the data source 501 comprises a set of measurements taken from Echocardiogram studies, however it will be readily apparent to the skilled addressee that the methods and systems disclosed below have application in imputing missing measurement data into records of different types of data sets (medical or otherwise) where a relatively large number of possible measurement data is included in each record.

A portion of the data records from data source 501 is designated as training data 503 which is used to train the AI system 500. Due to the complex nature of the human body, a large number of examples are typically required. Having a large number of example records (in the order of hundreds of thousands of records) better allows the AI model to distinguish between true patterns in the data and random noise caused by various factors such as human error. Approximately 60%-80%, typically about 70%, of the records from data source 501 is selected to form the training data. The remaining 20%-40%, typically about 30%, is designated as test data 505 which is used later in the process to validate the predictive capabilities of the AI model on a real data set with known data values. As will be appreciated by the skilled addressee, it is important not to use the training data 503 for AI model validation as many AI models are prone to overfitting-which would lead to the undesirable outcome of tailoring the model to patterns in the training data that are non-generalisable “noise” rather than meaningful data.

It is important to note that this architecture of system 500 allows the possibility of online-training—as more data is collected at a clinical site and continually added to the data source 501, the AI model may be continually refined using the data records to improve its predictive performance.

The AI models in the presently described arrangement of system 500 are provided with an initial model state 507 state which encodes the behaviour of the AI. For example, an Artificial Neural Network (ANN) model (a type of machine learning system) has a set of weight coefficients which are learned during the training process. A new model will typically be initialised with a random state, but in the case of online training, a previously trained model may be used as the initial model state.

As would be appreciated, the architecture 500 presented herein allows any AI model to be used in the system 500, provided that the inputs and outputs meet a standard interface specification which is particular to the desired outcomes of the system and taking into consideration the nature of the data records in data source 501. The details of the training process 509 largely depend on the AI algorithm selected, but typically algorithms will iteratively process each example and use the “mistakes” made by the model to “learn” by gradually mutating the model state. A successful training process will trend over time towards more accurate predictions.

After the training process 509 has completed, the AI model arrives at (converges to) a proposed trained model 511 for data analysis, which encodes the learned AI model state. Preferred training procedures 509 are non-parametric in that the training process methodology employed makes no explicit assumptions about the relationships between any of the variables or data elements in the data source 501. A useful analogy of the training process 509 would be training an AI-system (such as for example, system 500) to build a model of a cube across the three dimensions (i.e., the available variables or features to be modelled) of width, height and depth.

This is in contrast to traditional modelling techniques which assume an explicit parametric relationship or probabilistic model. Where, human trial-and-error is required to find relationships that are effective approximations. As the required degree of model fidelity increases this becomes difficult to the point of near-impossibility for a human when the number of variables is high and there are complex interdependences—the number of possibilities become too numerous for a human to try.

### MDN Model—Sparse-Data Mixture Density Network (MDN) Imputation Model

In alternative particular embodiments of system 500 the AI model takes the form of a sparse-data mixture density network (MDN) imputation model as discussed below (noting that any previous mathematical definitions in this document, in particular those definitions in relation to the Sparse-Data Self-Organising Map imputation model (A) discussed above, should be disregarded in favour of the definitions below).

Although the formulation discussed here for the sparse-data MDN may be applied to any choice of parametric mixture-model, for the purposes of demonstrating a concrete embodiment this section will provide additional details on the formulation of the algorithm using the choice of a Gaussian Mixture Model (GMM).

### MDN.1 Definitions

Let mₜ∈ [symbol] be the number of training examples.

Let nᵥ∈ [symbol] be the number of variables per training example.

Let N be the number of training epochs.

Let the “indicator matrix” be defined as:

$${I}_{i , j} = \{ \begin{matrix}0 & no recorded measurement for example i , variable j \\ 1 & measurement was recorded for example i , variable j\end{matrix}$$

Let the “retrieval function” for example i, variable j be defined as:

$$x ( i , j ) = \{ \begin{matrix}measured value & {I}_{i , j} = 1 \\ 0 & {I}_{i , j} = 0\end{matrix} i ∈ {ℤ}^{+} , j ∈ {ℤ}^{+} , i \leq {m}_{t} , j \leq {n}_{v}$$

Let the z-score normalisation constants be:

$${μ}_{j} = \frac{1}{{\sum }_{i = 1}^{{m}_{t}} {I}_{i , j}} {\sum }_{i = 1}^{{m}_{t}} x ( i , j ) \qquad ( B1 )$$

$${σ}_{j} = \sqrt{\frac{1}{{\sum }_{i = 1}^{{m}_{t}} {I}_{i , j}} {\sum }_{i = 1}^{{m}_{t}} {( x ( i , j ) - {μ}_{j} )}^{2}} \qquad ( B2 )$$

Let the z-score transformation function for variable j be:

$${z}_{j} ( x ) = \frac{x - {μ}_{j}}{{σ}_{j}}$$

Let a normalised example vector be:

$${x}_{i} = ( {z}_{1} ( x ( i , 1 ) ) , {z}_{2} ( x ( i , 2 ) ) , … , {z}_{{n}_{ν}} ( x ( {i}_{𝔯} {n}_{ν} ) ) ) \qquad ( B3 )$$

Let p_{holdout} be the probability that a variable is held out for use as a target output during the training process.

Let the random input partition vector for example i be:

$${Δ}_{i} = ( {δ}_{1} , {δ}_{2} , … , {δ}_{{n}_{v}} )$$

Let each δⱼ be drawn independent and identically distributed (i.i.d) from a single-trial binomial distribution:

$${δ}_{j} ∼ B ( 1 , 1 - {p}_{h o l d o u t} )$$

Let the negated partition vector be:

$$¬ {Δ}_{i} = ( 1 , 1 , … , 1 ) - {Δ}_{i}$$

Let the binary operator ⊙ represent element-wise vector multiplication:

$$x ⊙ y = ( {x}_{1} {y}_{1} , {x}_{2} {y}_{2} , … , {x}_{n} {y}_{n} ) \\ x , y ∈ {ℝ}^{n}$$

Let the training inputs be:

$${x}_{t r a i {n}_{i}} = {Δ}_{i} ⊙ {x}_{i}$$

Let the target outputs be:

$${x}_{{target}_{i}} = ¬ {Δ}_{i} ⊙ {x}_{i}$$

Let the neural net input sentinel vector be:

$${S}_{i} = 2 ( {I}_{i} ⊙ {Δ}_{i} ) - ( 1 , 1 , … , 1 ) {S}_{i} ∈ \{ - 1 , 1 \} \qquad ( 4 )$$

Let the mini-batch size be B.

Let the Gaussian probability density function (PDF) be defined as:

$$ϕ ( x ) = \frac{1}{\sqrt{2 π}} {e}^{- \frac{1}{2} {x}^{2}}$$

Let the Gaussian cumulative density function (CDF) be defined as:

$$ϕ ( x ) = {\int }_{- ∞}^{x} ϕ ( x ) dx$$

Let the soft max function be defined as:

$${softmax ( z )}_{j} = \frac{{e}^{{z}_{j}}}{{\sum }_{k = 1}^{K} {e}^{{z}_{k}}} \\ z ∈ {ℝ}^{K}$$

Let the neural network inputs for example i be:

$${x}_{{inp}_{i}} = ( {x}_{{train}_{i}} {S}_{i} )$$

Let a mini-batch of training data be:

$${x}_{batch} = ( \begin{matrix}{x}_{{inp}_{a}} \\ ⋮ \\ {x}_{{inp}_{a + B}}\end{matrix} ) ∈ {M}_{B \times 2 {n}_{v}} \qquad ( 5 )$$

where:
   1≤a≤mₜ−B is the starting index of the batch.

Let a mini-batch of target outputs be:

$${x}_{batch \_ target} = ( \begin{matrix}{x}_{{target}_{a}} \\ ⋮ \\ {x}_{{target}_{a + B}}\end{matrix} ) ∈ {M}_{B \times {n}_{ν}}$$

Let the batch partition matrix and negated batch partition matrix, respectively, be:

$${Δ}_{batch} = ( \begin{matrix}{Δ}_{a} \\ ⋮ \\ {Δ}_{a + B}\end{matrix} )$$

$$¬ {Δ}_{batch} = ( \begin{matrix}¬ {Δ}_{a} \\ ⋮ \\ ¬ {Δ}_{a + B}\end{matrix} )$$

Let c∈ [symbol] be the number of model mixture components.

Let nₚ∈ [symbol] be the number of parameters per mixture component.

Let the overall feedforward neural network function from inputs to outputs be:

$${f}_{n n} : {M}_{B \times 2 {n}_{v}} \to {M}_{B \times {n}_{ν} \times {n}_{p} c}$$

noting that fₙₙ may define any feedforward neural network with the given input and output dimensions but nominally 4 layers of width 2048 with the “leaky ReLU” activation function is sufficient.

Let the neural network output logits be:

$$logits = {f}_{n n} ( {x}_{batch} )$$

$$logits ∈ {M}_{B x {n}_{v} \times {n}_{p} c}$$

Let a parametric probability density function representing the chosen mixture model be:

$$\hat{f} ( x , {logits}_{i , j , 1} , … , {logits}_{i , j , {n}_{p} c} )$$

Let a parametric probability density function representing a probabilistic prediction for batch i, variable j be:

$${\hat{f}}_{ij} ( x ) = \hat{f} ( x , {logits}_{i , j , 1} , … , {logits}_{i , j , {n}_{p} c} )$$

Let the cumulative density function for the prediction for batch i, variable j be:

$${\hat{F}}_{ij} ( x ) = {\int }_{- ∞}^{x} {\hat{f}}_{ij} ( x ) dx$$

Let the continuous rank probability score (CRPS) of a probabilistic prediction for variable x with PDF \hat{f} (x) against the true value v be:

$$C R P S ( \hat{f} , v ) = {\int }_{- ∞}^{∞} {( \hat{f} ( x ) - v )}^{2} dx$$

Let the CRPS of the prediction for batch i, variable j be:

$$C R P {S}_{ij} = C R P S ( {\hat{f}}_{ij} , {x}_{{batch \_ target}_{ij}} )$$

Let the variable observation fractions be:

$$\overline{β} = \frac{1}{{m}_{t}} {\sum }_{i = 1}^{{m}_{t}} ( {I}_{i , 1} , … , {I}_{i , {n}_{v}} )$$

Let the variable observation correction factors be:

$$β = ( \frac{1}{{\overline{β}}_{1}} , … , \frac{1}{{\overline{β}}_{{n}_{v}}} )$$

Let the batch loss function be:

$$loss ( {x}_{batch} , {x}_{batch \_ target} ) = \frac{1}{B} {\sum }_{i = 1}^{B} \frac{1}{1 + {\sum }_{j = 1}^{{n}_{v}} ¬ {Δ}_{{batch}_{ij}}} {\sum }_{j = 1}^{{n}_{v}} {β}_{j} ( ¬ {Δ}_{{batch}_{ij}} ) C R P {S}_{ij}$$

For the case where the chosen mixture model is a GMM, let nₚ=3 and the neural network output logits be:

$$( \begin{matrix}{logit}_{π} \\ {logit}_{μ} \\ {logit}_{σ}\end{matrix} ) = logits$$

$${logit}_{π} , {logit}_{μ} , {logit}_{σ} ∈ {M}_{B \times {n}_{v} \times c}$$

For the case where the chosen mixture model is a GMM, let ϵ_{σ}>0 be a small constant setting the minimum standard deviation on a mixture component to avoid issues caused by limited numeric precision.

For the case where the chosen mixture model is a GMM, let the output predictions be:

$${π}_{ijk} = {softmax ( ( {logit}_{{π}_{ij 1}} , … , {logit}_{{π}_{ijk}} ) )}_{k} \\ {μ}_{ijk} = {logit}_{{μ}_{ijk}} \\ {σ}_{ijk} = \{ \begin{matrix}{e}^{{logit}_{{σ}_{ijk}}} + {ϵ}_{σ} & {logit}_{{σ}_{ijk}} < 0 \\ {logit}_{{σ}_{ijk}} + 1 + {ϵ}_{σ} & {logit}_{{σ}_{ijk}} \geq 0\end{matrix}$$

For the case where the chosen mixture model is a GMM, let:

$${\hat{f}}_{i j} ( x , {logits}_{i , j , 1} , … , {logits}_{i , j , {n}_{p} c} = {\sum }_{k = 1}^{c} {π}_{i j k} ϕ ( \frac{x - {μ}_{ijk}}{{σ}_{i j k}} )$$

For the case where the chosen mixture model is a GMM, note that:

$${\hat{F}}_{i j} ( x ) = {\sum }_{k = 1}^{c} {π}_{i j k} Φ ( \frac{x - {μ}_{ijk}}{{σ}_{ijk}} )$$

For the case where the chosen mixture model is a GMM, note that there exists a closed form solution:

$$CRP {S}_{i j} = {\sum }_{k = 1}^{c} {π}_{i j k} A ( {x}_{{batch \_ target}_{ij}} - {μ}_{i j k} , {σ}_{ijk}^{2} ) - \frac{1}{2} {\sum }_{k = 1}^{c} {\sum }_{l = 1}^{c} {π}_{i j k} {π}_{ijt} A ( {μ}_{i j k} - {μ}_{i j l} , {σ}_{ijk}^{2} + {σ}_{i j l}^{2} ) \\ where : \\ A ( μ , {σ}^{2} ) = 2 σ ϕ ( \frac{μ}{σ} ) + μ ( 2 Φ ( \frac{μ}{σ} ) - 1 )$$

### MDN.2 Training Algorithm

### MDN.2.1 Inputs

1. A partially observed set of mₜ×nᵥ measurements.
2. Chosen values for parameters: N, c, B, ϵ_{σ}, p_{holdout}
3. Neural network parameters: number of nodes per layer, number of layers, optimiser parameters.

### MDN.2.2 Initialisation

1. Duplicate the normalised example vectors N times and randomly shuffle to create a new sequence of vectors and corresponding indicator vectors:

$${x}_{1} , {x}_{2} , … , {x}_{N {m}_{t}} \\ {I}_{1} , {I}_{2} , … , {I}_{N {m}_{t}}$$

2. Initialise the neural network weights and biases using the desired technique (nominally the Glorot uniform initializer performs well).
3. Initialize the optimizer algorithm.

### MDN.2.3 Main Algorithm

For each batch x_{batch}, x_{batch_target} taken in sequence from x₁, x₂, . . . , x_{Nm}_{ₜ}.
1. Calculate loss(x_{batch}, x_{batch_target}).
2. Using automatic differentiation, calculate the loss gradient with respect to each neural network weight:

$$\frac{\partial }{\partial w} loss ( {x}_{batch} , {x}_{batch \_ target} ) .$$

3. Update the neural network using backpropagation with the chosen optimizer algorithm.

### MDN.2.4 Outputs

1. Trained neural network.
2. Normalisation constants μ₁, . . . μₘ, σ₁, . . . σₙ_{ₚ}.

### MDN.3 Imputation Algorithm

The imputation algorithm disclosed below provides the system 500 with the ability to fill in missing or blank data measurement fields from records in the data source 501 during model validation 513 and also to predict missing or additional data measurements in production data source 519 whilst the system is in use. Generally speaking, imputation preserves all cases by replacing missing data with an estimated value based on other available information. In the algorithm below, the estimated values for missing data are provided as probability distributions of likely values.

### MDN.3.1 Inputs

1. Trained neural network.
2. Normalisation constants.

$${μ}_{1} , … {μ}_{{n}_{v}} , {σ}_{1} , … {σ}_{{n}_{v}}$$

3. Normalised output of the retrieval function for a set of measurements (as discussed in Paragraph [0150] (above):

$$x ∈ {𝕄}^{B \times {n}_{v}}$$

4. The indicator matrix for the batch of measurements:

$$I ∈ {\{ 0 , 1 \}}^{B \times {n}_{ν}}$$

### MDN.3.2 Main Algorithm

1. Redefine the partition vector at inference time to be a constant Δᵢ=(1, 1, . . . , 1).
2. Build x_{batch} using x and I as per the definition provided in Paragraph above.
3. Run forward inference on the neural network to derive probabilistic imputations \hat{f}ᵢⱼ, \hat{F}ᵢⱼ.

### MDN.3.3 Outputs

1. Imputed density functions \hat{f}ᵢⱼ, \hat{F}ᵢⱼ.

The classification algorithm disclosed below provides the system 500 with the ability to classify the likely severity of disease in a patient based on the imputed density function for a given measurement and established clinical thresholds for that measurement, from records in the data source 501 during model validation 513 and from measurements in production data source 519 whilst the system is in use.

### MDN.4 Classification Algorithm

### MDN.4.1 Inputs

1. Imputed mu's and sigma's of the density functions \hat{f}ᵢⱼ, \hat{F}ᵢⱼ.
2. A choice of threshold variable j.
3. A set of T−1 clinical thresholds for variable j defining T gradings of disease severity (e.g., normal, mild, moderate, severe):

$${t}_{1} … {t}_{T - 1} \\ or \\ {t}_{1} = threshold\_var \_mid or {t}_{2} = threshold\_var \_high$$

### MDN.4.2 Main Algorithm

For the predicted AVA, evaluate its cumulative distribution function (CDF) value on the 1.0 AVA value. If the CDF value is greater than threshold_var_mid and less than threshold_var_high, the phenotype is moderate AS. If the CDF value is greater than threshold_var_high, the phenotype is severe AS.

### MDN.4.3 Outputs

### AS Phenotype

### EXAMPLES

An example of a dataset that replicates a sparsely populated data source 501 is shown in Table 1 below.

TABLE 1
Example Sparsely Populated Data Source

| Width (cm) | Height (cm) | Depth (cm) |
|---|---|---|
| 1 | 1 | 1 |
| 13 | | 13 |
| | 21 | 21 |
| 0.1 | 0.1 | |

Using data source of Table 1 as a training set 503, the system is configured to develop a model of a cube which is constrained by the fact that width=height=depth. Once the model is learnt then predictions can be made on similarly sparsely populated data sets. For example, if the width is known then both the height and depth of the cube can be predicted. This simple example demonstrates how the blank data fields of the cube records are filled in the original sparse dataset i.e., see Table 2 below comprising the initial data set (normal font typeset) and predicted measurements (boldface typeset) output from the trained AI model.

TABLE 2
Example Sparsely Populated Data Source with Imputed Data

| Width (cm) | Height (cm) | Depth (cm) |
|---|---|---|
| 1 | 1 | 1 |
| 13 | 13 | 13 |
| 21 | 21 | 21 |
| 0.1 | 0.1 | 0.1 |

In the embodiments described herein, the dataset of a cube (Table 1) is substituted by the NEDA database which allows a model (phenotype) of a heart to be built across several variables. This phenotype model data describes the interrelation of variables across many different heart configurations and diseases.

Model validation 513 makes use of the test data 505 to evaluate the performance of the trained AI model 511 on new, previously unseen data. The outputs of the validation process 513 largely depend on the particular application and the data source 501 used which are tested against particular performance metrics 515 to evaluate the predictive power (e.g., the probability that a predicted data measurement will be within defined tolerance levels as compared with the actual measurement data). An example of a useful performance metric 515 is Root-Mean-Square Error of predicted measurements compared with known data in the test data 505.

Once the trained model 511 is validated to be able to provide meaningful measurement predictions within pre-defined tolerance levels (defined according to typical measurement error ranges taken from validation studies plus a tolerance of about 5%) the trained model 511 is imported to the primary predictive engine 517 of system 500. The prediction engine 517 is then used to analyse new data e.g., production data set 519, during system operation which may be derived directly from measurements made by a sonographer of a patient during an echocardiographic examination. For example, e.g., production data set 519, may be a database of measurements in an echo reporting software package, or it may be measurements on an echo/ultrasound workstation obtained during working of methods 200 or 300 disclosed above. The outputs from the AI prediction engine 517 comprise predicted measurements 521 and/or predicted diagnoses 523 with respect to the production data source 519, for example, obtained during working of methods 200 or 300 disclosed above. As will be readily appreciated, the outputs of the AI prediction engine 517 may be a set of predictions for measurements 521 that were not provided as inputs or alternatively a predicted diagnosis on the basis of a set of pre-defined risk factors for various diseases relevant to the nature of the data source, for example where the data source comprises echocardiography measurements, the AI prediction engine 517 may provide predictions of a particular patient's probability of possessing, or likely to subsequently possess, heart-related diseases such as, for example arterial stenosis or heart chamber or valve malfunction.

AI prediction engine 517 may also utilise a tailored implementation of the AI algorithm to produce predictions in the required operating (e.g., software) environment. For example, an implementation for an echo software package would likely be implemented to run on Microsoft Windows, whereas an implementation for an echo workstation would need to be tailored to the vendor-specific hardware and operating system running on the workstation.

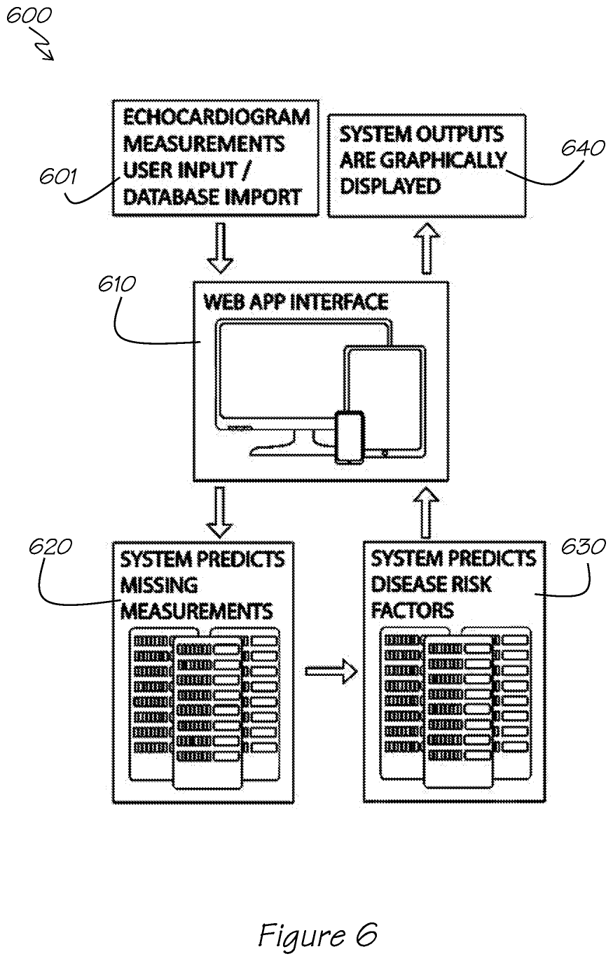

A further example embodiment of the systems disclosed above may be implemented as an application which is made available to, for example, healthcare professionals, over a communications network such as, for example, a diagnostic clinic intranet network or a publicly accessible communications network such as the internet. The network-accessible application may be provided as an interactive web application with a simple workflow, such as depicted in an example wireframe schematic representation 600 shown in FIG. 6. In an example workflow embodiment of application 600 in use, a user enters a set of echo measurements (or alternatively connects the input to a database of echo measurements e.g., for a plurality of patients) into an input interface 601 of web interface 610 (where the application 600 is accessible over the worldwide web/internet). The backend of application 600 (not shown) is connected to an AI system such as system 500 depicted in FIG. 5. Application 600 feeds the inputted user data to the backend analysis system so as to return outputs comprising predicted measurement data 620 to fill in any blank or missing data measurements from the input data and further to output predicted disease risk factor 630. Predicted measurements and disease risk factors are then presented to the user via interface 610 to a user display means 640, which may present the output predictions in any useful manner for interpretation by the user, for example the outputs may be presented in a graphical form for easy interpretation by the user.

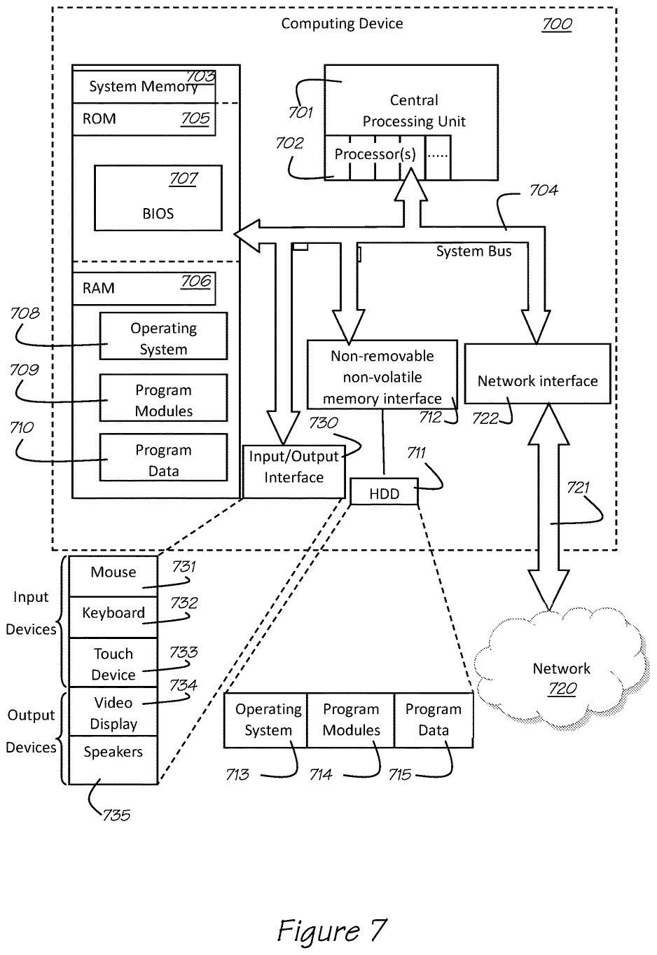

The methods of training and operating an AI-assisted echocardiography system as disclosed herein (e.g., methods 200, 300, 400 and 500 depicted in FIGS. 2, 3, 4 and 5 respectively may be implemented using a computer system 700, such as the example computer system shown in FIG. 7 with which embodiments described herein may be implemented wherein the processes of FIGS. 2 to 5 may be implemented as software, such as one or more application programs executable within the computing device 700. In particular, the steps of method(s) 200, 300, 400 and 500 are effected by instructions in the software that are carried out within the computer system 700. The instructions may be formed as one or more code modules, each for performing one or more particular tasks. The software may also be divided into two separate parts, in which a first part and the corresponding code modules performs the described methods and a second part and the corresponding code modules manage a user interface between the first part and the user. The software may be stored in a computer readable medium, including the storage devices described below, for example. The software is loaded into the computer system 700 from the computer readable medium, and then executed by the computer system 700. A computer readable medium having such software or computer program recorded on it is a computer program product. The use of the computer program product in the computer system 700 preferably effects an advantageous apparatus for AI assisted echocardiography.

In the example of FIG. 7, example computer system 700 and instructions for implementing the disclosed technologies in hardware, software, or a combination of hardware and software, are represented schematically, for example as boxes and circles, at the same level of detail that is commonly used by persons of ordinary skill in the art to which this disclosure pertains for communicating about computer architecture and computer systems implementations.

The example computing device 700 can include, but is not limited to, one or more central processing units (CPUs) 701 comprising one or more processors 702, a system memory 703, and a system bus 704 that couples various system components including the system memory 703 to the processing unit 701. The system bus 704 may be any of several types of bus structures including a memory bus or memory controller, a peripheral bus, and a local bus using any of a variety of bus architectures. The computing device 700 also typically includes computer readable media, which can include any available media that can be accessed by computing device 700 and includes both volatile and non-volatile media and removable and non-removable media. By way of example, and not limitation, computer readable media may comprise computer storage media and communication media. Computer storage media includes media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by the computing device 700. Communication media typically embodies computer readable instructions, data structures, program modules or other data in a modulated data signal such as a carrier wave or other transport mechanism and includes any information delivery media. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 703 includes computer storage media in the form of volatile and/or non-volatile memory such as read only memory (ROM) 705 and random-access memory (RAM) 706. A basic input/output system 707 (BIOS), containing the basic routines that help to transfer information between elements within computing device 700, such as during start-up, is typically stored in ROM 705. RAM 706-typically contains data and/or program modules that are immediately accessible to and/or presently being operated on by processing unit 701. By way of example, and not limitation, FIG. 7 illustrates an operating system 708, other program modules 709, and program data 710.

The computer readable instructions stored in memory 703, ROM 705, RAM 706 or HDD storage 711 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

The computing device 700 may also include other removable/non-removable, volatile/non-volatile computer storage media. By way of example only, FIG. 7 illustrates a hard disk drive 711 that reads from or writes to non-removable, non-volatile magnetic media. Other removable/non-removable, volatile/non-volatile computer storage media that can be used with the example computing device include, but are not limited to, magnetic tape cassettes, flash memory cards, digital versatile disks, digital video tape, solid state RAM, solid state ROM, and the like. The hard disk drive 711 is typically connected to the system bus 704 through a non-removable memory interface such as interface 712.

The drives and their associated computer storage media discussed above and illustrated in FIG. 7, provide storage of computer readable instructions, data structures, program modules and other data for the computing device 700. In FIG. 7, for example, hard disk drive 711 is illustrated as storing an operating system 713, other program modules 714, and program data 715. Note that these components can either be the same as or different from operating system 708, other program modules 709 and program data 710. Operating system 3013, other program modules 714 and program data 715 are given different numbers hereto illustrate that, at a minimum, they are different copies.

The computing device also includes one or more input/output (I/O) interfaces 730 connected to the system bus 704 including an audio-video interface that couples to output devices including one or more of a video display 734 and loudspeakers 735. Input/output interface(s) 730 also couple(s) to one or more input devices including, for example a mouse 731, keyboard 732 or touch sensitive device 733 such as for example a smartphone or tablet device. In the embodiments disclosed herein, input interface 730 may also comprise an echocardiography/ultrasound handpiece and computing device 700 may comprise or be integrated with an echo/ultrasound workstation.

Of relevance to the descriptions below, the computing device 700 may operate in a networked environment using logical connections to one or more remote computers. For simplicity of illustration, the computing device 700 is shown in FIG. 7 to be connected to a network 720 that is not limited to any particular network or networking protocols, but which may include, for example Ethernet, Bluetooth or IEEE 802.X wireless protocols. The logical connection depicted in FIG. 7 is a general network connection 721 that can be a local area network (LAN), a wide area network (WAN) or other network, for example, the internet. The computing device 700 is connected to the general network connection 721 through a network interface or adapter 722 which is, in turn, connected to the system bus 704. In a networked environment, program modules depicted relative to the computing device 700, or portions or peripherals thereof, may be stored in the memory of one or more other computing devices that are communicatively coupled to the computing device 700 through the general network connection 721. It will be appreciated that the network connections shown are example and other means of establishing a communications link between computing devices may be used.

All analyses discussed herein are derived from echocardiograms performed from Apr. 11, 2000 to Jun. 13, 2017. They involved 530,871 echocardiograms from 331,344 adults aged >18 years (52% male) with follow up mortality data up until 30/10/2017 (study census).

### Example 1—Aortic Stenosis Diagnosis

The following examples demonstrate the utility of the AI-assisted echocardiography methods and systems disclosed above. The methods are used to predict the incidence of aortic stenosis while completely removing the need for left ventricular outflow tract measurements by the use of artificial intelligence. It is observed that the above disclosed systems and methods improve the consistency of echo, in addition to saving a significant amount of scanning time for the sonographer performing echocardiography studies.

Comprehensive evaluation of the aortic valve is a standard part of every echo examination, requiring measurements performed from multiple echo windows and the use of 2-dimensional measurements and spectral Doppler. Measurement of aortic velocities using continuous wave Doppler is accurate and reproducible, but the same measurements in the LVOT are prone to error. Any error in 2D measurement of the LVOT is magnified by multiplying and squaring the measurement as part of the continuity equation (CE). The time required for aortic valve area calculation is approximately 7 minutes per patient.

The goal of the models is to produce a comprehensive echo interpretation system using artificial intelligence to provide efficient, fast, reproducible echo examinations with accurate and reliable interpretation. Within this larger project it was evaluated whether AI could impute the aortic valve area from other echo data, with the aim of producing a system just as accurate as the traditional aortic valve area calculation, but more reproducible and faster with less images and measurements.

### Guidelines

The current clinically accepted guidelines for diagnosis of severe aortic stenosis (AS) in a patient recommend that the following three main criteria are used:

$$AS Jet Velocity >= 4. m / s \\ AV Mean Gradient >= 40 mmHg \\ AV Area = < 1 {cm}^{2}$$

Due to the complex geometry of the aortic valve, rather than directly measuring its cross-sectional area, an estimate of its effective orifice area is calculated using the following equations:

$$LVOT Area = π \times {( 1 / 2 \times LVOT Diameter )}^{2} \qquad ( 3 )$$

Where LVOT diameter is a circular approximation of cross-sectional area measured by the sonographer during an echocardiogram study of a patient used in evaluation of the continuity equation (CE) above.

### Difficulties in Diagnosing AS

It is important to note that AS Jet Velocity and AV Mean Gradient are dependent on flow rate and so in patients with low flow, a low value for these measurements does not rule out the possibility of Aortic Stenosis.

The estimated AV Area is relatively independent of flow and so in patients with low flow, it becomes a critical parameter. However, it is not without its own difficulties due to its dependence on the estimated LVOT Area. To quote the current guidelines “[ . . . ] the measurement variability for LVOT diameter ranges from 5 to 8%. When LVOT diameter is squared for the calculation of CSA, it becomes the greatest potential source of measurement error in the continuity equation”.

For example, in a patient with an LVOT diameter of 2 cm, pi*(2/2)\hat{ }2=pi=3.14 cm² and pi*2.1*2.1/4=1.1 pi=3.45 cm², therefore LVOT VTI=20 cm and AV VTI=40 cm. The Aortic valve (AV) cross sectional area is then given by:

$$AV Area = LVOT Area * LVOT VTI / AV VTI \qquad ( 4 )$$

such that A1=3.14*20/40=1.57 cm² and A2=3.45*20/40=1.72 cm². Now, say LVOT VTI=15 cm, AV VTI=45 cm, therefore:

$$A 1 = 3 \text{.14} / 3 = 1.05 moderate \\ and \\ A 2 = 3 \text{.45} / 3 = 1.15 mild$$

### Example 1A—AS Prediction in General Population

### Procedure

1. A snapshot of the NEDA database (approx. 650,000 patients) including patient mortality data, for a wide range of cardiac disease states of the type normally associated with diagnosis via echochardiographic diagnostic procedures was taken and split into a 70% training set and a 30% test set.
2. The training set was used to train an imputation model using the above-disclosed MDN algorithm.
3. The training set was also used to train the AI engine to associate phenotype characteristics with one or more associated disease states including, among others, aortic stenosis.
4. The test set was subsampled to only include patients with a complete set of measurements for:
   AS Jet Velocity (A.K.A. AV Peak Velocity)
   AV Mean Gradient
   LVOT Diameter
   LVOT VTI
   AV VTI
5. The AV Area was calculated for each patient in the subsampled test set using equation (4) above and a “ground truth” binary label was generated for each patient in the subsampled test set, labelling them as either “Severe AS” or “Not Severe AS”. The patients were labelled as “Severe AS” if and only if AV Area <1 cm².
6. The measured values for “LVOT Diameter” and “LVOT VTI” were deleted from each patient. In addition, the values for “LVOT Mean Velocity” and “LVOT Peak Velocity” were deleted as these are highly correlated with LVOT VTI.
7. The imputation model disclosed above was used to predict values for “LVOT Diameter” and “LVOT VTI” in place of the deleted values.
8. Predicted AV Area was calculated for each patient using inputs of: Predicted LVOT DiameterPredicted LVOT VTIMeasured AV VTI.
9. Patient records are associated with and classified by phenotype characteristics of the patient's associated measurement data.
10. A Receiver Operating Characteristic (ROC) plot and a Precision-Recall plot were generated to study the effectiveness of the predicted AV Area as a predictor of Severe AS at different cutoff values. The “ground truth” labels from step 4 were used.
11. Patient's classified phenotype characteristics are then associated with one or more disease states to predict a probable disease state in the patient.

The measurements included in the imputation model include AR Peak Velocity; AR Pressure Half Time; AV Mean Gradient; AV Mean Velocity; AV Peak Velocity; AV Velocity Time Integral; Aorta at Sinotubular Diameter; Aorta at Sinuses Diameter; Aortic Root Diameter; Aortic Root Diameter MM; Ascending Aorta Diameter; Body Weight; Diastolic Slope; Distal Transverse Aorta Diameter; EFtext; Heart Rate; IVC Diameter Expiration; IVS Diastolic Thickness; IVS Diastolic Thickness MM; IVS Systolic Thickness MM; LA Area 4C View; LA Length 4C; LA Systolic Diameter LX; LA Systolic Diameter MM; LA Systolic Diameter Transverse; LV Diameter; LV Diastolic Area 2C; LV Diastolic Area 4C; LV Diastolic Area PSAX; LV Diastolic Diameter 4C; LV Diastolic Diameter MM; LV Diastolic Diameter PLAX; LV Diastolic Length 2C; LV Diastolic Length 4C; LV E′ Lateral Velocity; LV Epi Diastolic Area PSAX; LV Mass (C) d; LV Relative Wall Thickness; LV Systolic Area 2C; LV Systolic Area 4C; LV Systolic Diameter 4C; LV Systolic Diameter Base LX; LV Systolic Diameter MM; LV Systolic Diameter PLAX; LV Systolic Length 2C; LV Systolic Length 4C; LVOT Diameter; LVOT Mean Velocity; LVOT Peak Velocity; LVOT Velocity Time Integral; LVPW Diastolic Thickness; LVPW Diastolic Thickness MM; LVPW Systolic Thickness MM; MV A′ Velocity; MV Deceleration Time; MV E′ Velocity; MV Mean Velocity; MV Peak Velocity; MV Pressure Half Time; MV Velocity Time Integral; Mitral A Point Velocity; Mitral E Point Velocity; PV Peak Velocity; PV Velocity Time Integral; PatientAge; Pulm Vein Atrial Duration; Pulm Vein Atrial Reversal Velocity; Pulmonary Vein A Velocity; Pulmonary Vein Diastolic Velocity; Pulmonary Vein Systolic Velocity; RA Area; RA Systolic Diameter LX; RA Systolic Diameter Transverse; RApressuretext; RVOT Peak Velocity; Right Atrial Pressure; TR Peak Velocity; and Thoracic Aorta Diameter.

### Results

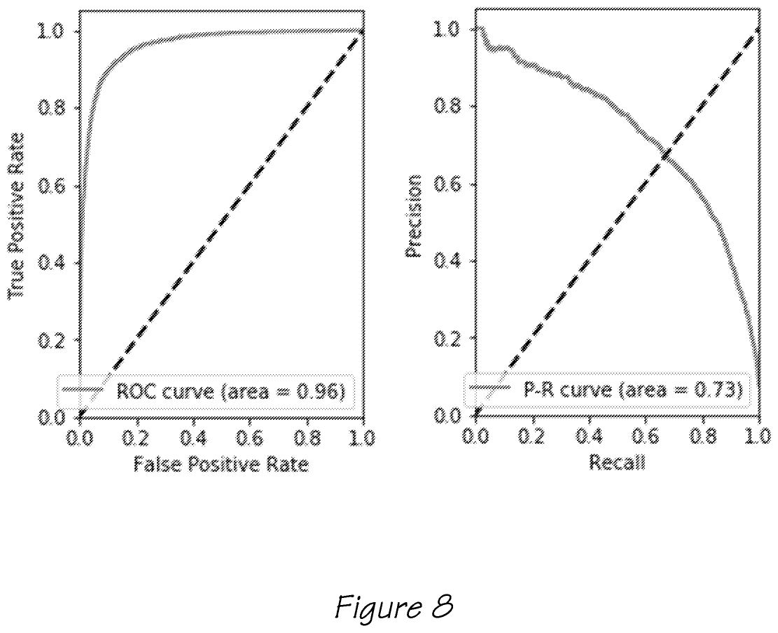

FIG. 8 shows the Receiver Operating Characteristic and Precision-Recall curve area for this example. The total patients in the sample was 24748 and the diagnosis was based on the following measurements: AV Area (VTI), Holding out: [“LVOT Mean Velocity”, “LVOT Peak Velocity”, “LVOT Velocity Time Integral”, and “LVOT Diameter”]. The number of patients predicted with severe AS was 1834 (7.410700%) and the number of patients predicted without severe was 22914 (92.589300%).

### Example 1B—AS Prediction in Patients with Reduced Ejection Fraction

Because of the potential the general population results may have been influenced by the larger number of patients with normal LV systolic function, and possibly masked a poorer performance in the setting of impaired LV function, the experiment above was repeated for those patients having an EF<50% and <30% as discussed below.

### Procedure

1. Follow all instructions for Experiment 1, except at step 3, subsample to only include patients with Ejection fraction <=50%.
2. Repeat step 1 for ejection fractions of 40%, 35%, 30%.

### Results

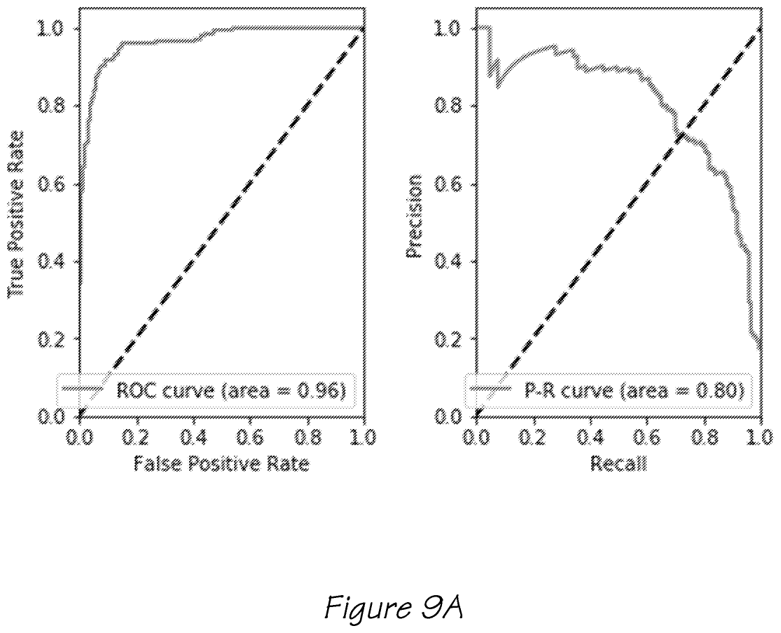

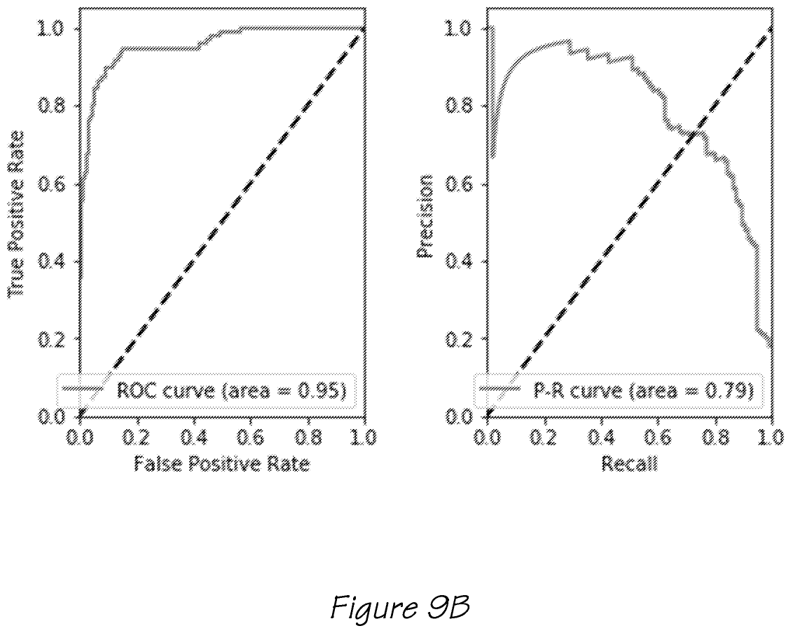

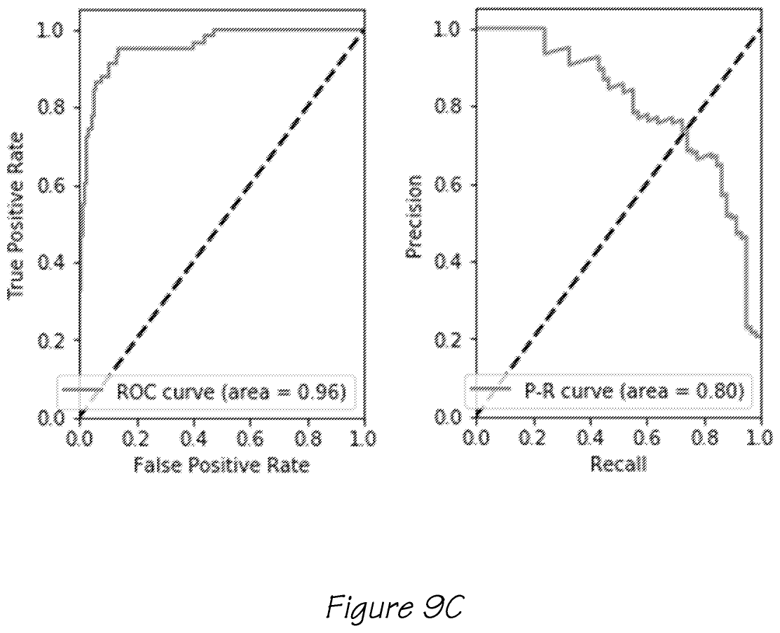

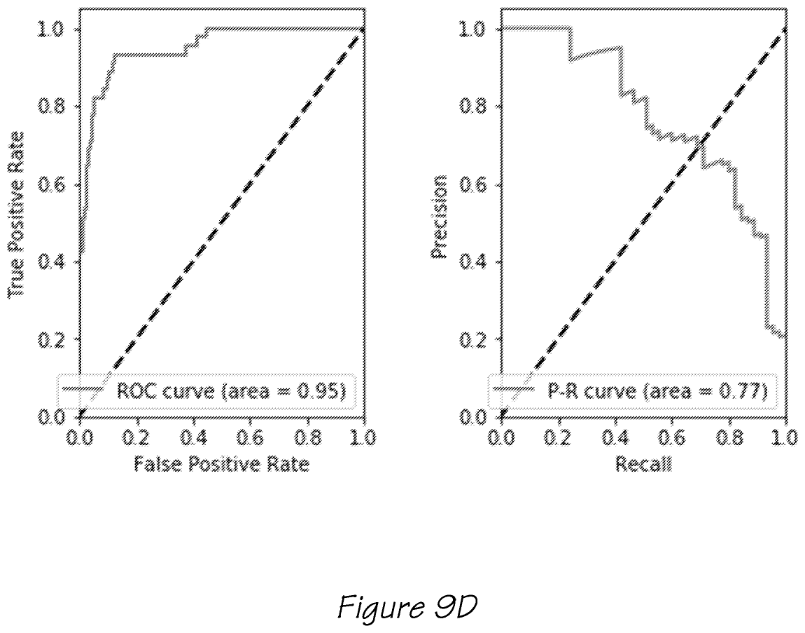

FIG. 9A shows the results of the model using an ejection fraction value of <=50%, and Diagnosing based on: AV Area (VTI), Holding out: [“LVOT Mean Velocity”, “LVOT Peak Velocity”, “LVOT Velocity Time Integral”, “LVOT Diameter”] and removing patients with EF >50.000000. The total number of patients in the sample was 1391. The number of patients predicted with severe AS was 143 (10.280374%) and the number of patients predicted without severe was 1248 (89.719626%)

FIG. 9B shows the results of the model using an ejection fraction value of <=40%, and diagnosing based on: AV Area (VTI), Holding out: [“LVOT Mean Velocity”, “LVOT Peak Velocity”, “LVOT Velocity Time Integral”, “LVOT Diameter”] and removing patients with EF >40.000000. The total patients in the sample was 861. The number of patients predicted with severe AS was 96 (11.149826%) and the number of patients predicted without severe AS was 765 (88.850174%).

FIG. 9C shows the results of the model using an ejection fraction value of <=35%, and diagnosing based on: AV Area (VTI), Holding out: [“LVOT Mean Velocity”, “LVOT Peak Velocity”, “LVOT Velocity Time Integral”, “LVOT Diameter”] and removing patients with EF >35.000000. The number of patients predicted with severe AS was 58 (11.026616%) and the number of patients predicted without severe AS was 468 (88.973384%).

FIG. 9D shows the results of the model using an ejection fraction value of <=30%, and diagnosing based on: AV Area (VTI), Holding out: [“LVOT Mean Velocity”, “LVOT Peak Velocity”, “LVOT Velocity Time Integral”, “LVOT Diameter”] and removing patients with EF >30.000000. The total patients in the sample was 426. The number of patients predicted with severe AS was 45 (10.563380%) and the number of patients predicted without severe AS was 381 (89.436620%).

### Example 1—Results Summary

Table 3 below summarises the results of Examples 1A and 1B above.

TABLE 3
Result Summary-Prediction of Aortic Stenosis

| | | Percentage of | | |
|---|---|---|---|---|
| Experiment | Sample | Sample with | | Avg. |
| Name | Size | Severe AS (%) | AUC | Precision |
| General | 24748 | 7.41 | 0.96 | 0.73 |
| Population | | | | |
| EF <= 50% | 1391 | 10.28 | 0.96 | 0.80 |
| EF <= 40% | 861 | 11.14 | 0.95 | 0.79 |
| EF <= 35% | 526 | 11.02 | 0.96 | 0.80 |
| EF <= 30% | 426 | 10.56 | 0.95 | 0.77 |

### Example 1—Discussion

Average precision is generally a more informative metric than AUC for evaluating binary classifier performance with imbalanced data. With this in mind, the results are interpreted as follows:

From Table 3, it can be seen that Severe AS can be predicted in the general population with AUC=0.96 and average precision of 73% without measurements for LVOT Diameter or LVOT VTI. In the subpopulation of patients with ejection fraction <=50%, the average precision improves to 80% and the other subpopulations of patients with reduced EF have similar results. This is particularly significant, given that in these subpopulations the imputation model cannot necessarily rely on simple associations with elevated AV Mean Gradient/AV Peak Velocity. Future work will investigate which variables are the major contributors to the surprising performance of the imputation model in this setting.

Some important points to note:
The sample sizes for the reduced-EF subpopulations are an order of magnitude smaller than the general population. Hence, there is more uncertainty associated with the resulting AUC and avg. precision statistics. However, a sample size of 1391 patients is still reasonably large.
The prevalence of Severe AS is higher in the reduced-EF subpopulations. Precision-Recall is relatively insensitive to a shift in class distribution so the average precisions may be meaningfully compared. But more investigation is still required to determine the exact reasons for the improved performance on this subpopulation. For example, it could be that the “decision boundary” in this subpopulation is more pronounced due to more advanced progression of disease.

Evaluation of AS severity in severe LV dysfunction is notoriously difficult because of low flows and resultant low gradients. It was found that in patients with EF<50% the ROC area was 0.96, P-R area 0.80 (in 1391 studies, 10% with severe AS). In EF<30% the ROC area was 0.85, P-R area 0.77 (in 426 studies, 10% with severe AS).

This confirmed that our model identified severe AS just as well in impaired LV function as in normal LV function.

### Example 1—Comparison Between ROC and Precision-Recall Curves on Data with Imbalanced Classes

The Receiver Operating Characteristic (ROC) curve plots true-positive-rate (TPR) versus false-positive-rate. (FPR). The Precision-Recall curve plots precision versus recall. The relevant equations are:

$$TPR = Recall = TP / ( TP + FN ) \\ FPR = FP / ( FP + TN ) \\ Precision = TP / ( TP + FP ) \\ where : \\ TP = True Positives \\ FP = False Positives \\ TN = True Negatives \\ FN = False Negatives$$

In the setting of the examples above, the metrics can be described as follows:
Recall/TPR is the percentage of patients with severe AS that the system correctly identifies.
FPR is the percentage of patients without severe AS that the system incorrectly diagnoses with severe AS.
Precision is the fraction of positive diagnoses for severe AS generated by the system that are correct.

Hence, Precision-Recall is a better metric with an imbalanced test set for the following reasons:
The FPR metric is relative to the number of patients without severe AS, which in this setting is the majority of patients. Hence an FPR that seems low actually translates to a large number of false positive diagnoses.
The precision metric is relative to the number of positive diagnoses generated by the algorithm. This gives a better sense of how the system would be perceived in actual usage. For example, a precision of 80% means that if the system diagnoses a patient with severe AS then there is an 80% chance it is correct.

### Example 2—Severe Aortic Stenosis

Given the size and scope of available data no formal analyses of statistical power were performed. After training the AI system, the two AS severity predictions from the 30% test data set (comprehensive and limited echocardiography), along with the de-identified copy of the NEDA database, was imported into the Statistical Package for Social Sciences (SPSS) version 25.0, IBM Corporation, for comparisons with the continuity-derived aortic valve area. Statistical significance was set at a P-value <0.05. A probability cut-off was chosen for the severe AS prediction using AI for both the comprehensive and limited echocardiography, optimized for approximately equal sensitivity and specificity, and used for the remainder of the analysis. Discrete variables were presented as numbers and percentages. Between-group comparisons were assessed by Student's t-tests, Mann Whitney U test (for non-normally distributed continuous data), and Chi-squared tests (for categorical data).

Big data from the National Echocardiography Database of Australia was used to create a modified mixture density network AI system. 530,000 echocardiograms on 331,344 adult individuals with follow-up mortality data were randomly split into 363,887 studies (70%) used to train the AI, and 155,967 studies (30%) used to verify the performance of the trained model. Two models were trained to predict severe AS without the need for left ventricular outflow tract data, one using the remainder of the echocardiographic measurements and the other using a limited data set, likely to change with aortic stenosis.

Performance of the AI system was assessed using receiver operating characteristic (ROC) curves, with the prediction of severe AS by AI against the aortic valve area calculated using the continuity equation. The area under the ROC (AUC) curve was calculated for all patients and for those with impaired left ventricular function. All fatal events were identified, and the last recorded echocardiogram for those patients with complete follow up used to calculate actuarial 5-year mortality survival curves. Cox proportional hazard ratios adjusted for age and gender were calculated, along with further adjustments for the aortic valve area and mean aortic gradient. To establish potential differences in survival of those with AI-diagnosed severe AS compared those with severe AS using calculated aortic valve area, actuarial 5-year survival curves were examined. The phenotypic diagnosis of severe AS were compared using the AI system to that of the continuity-derived severe AS.

To assess whether the results were predominantly a result of patients with classic high gradient, normal ejection fraction severe AS, three groups of patients (full system and limited data-set) were compared: 1) All patients in the test set, 2) those with an ejection fraction <50% and 3) those with an ejection fraction <30%. Patients without a full set of measurements available for calculation of the continuity equation were excluded from statistical analysis (n=361,067 patients without a valid left ventricular outflow tract measurements). The AI prediction of severe AS used all available data (minus the left ventricular outflow tract data), with missing measurements ignored by the AI. For the limited data set, only the measurements specified above were visible to the AI (and only if measured). The aortic valve area was then imputed by the AI system based on the remainder of the available echo measurements, followed by calculation of a probability of severe AS, as described above.

### Example 2—Results

In the test set, 2382 of 32,574 individuals (7.38%) with a complete set of measurements had severe aortic stenosis, predicted by the AI system with an area under the receiver operating characteristic curve (AUC) of 0.97. The AUC was 0.95 in patients with a left ventricular ejection fraction <50%, and 0.92 in those with ejection fraction <30%. The performance of the AI was maintained using the limited data set (AUC 0.97, 0.94, and 0.93 respectively).

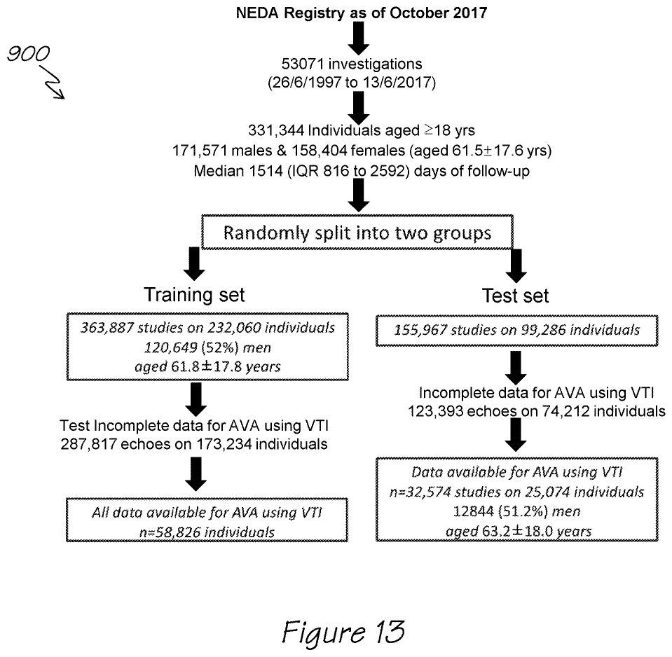

FIG. 13 shows the flow 900 of analyses performed on data derived from the study cohort of 171,571 males & 158,404 females (aged 61.5±17.6 years) with a median 4.1 (IQR 2.2, 7.1 years) follow. There were no differences in the baseline characteristics between the 70% test set and the 30% training set, nor for those with a complete set of data available for aortic valve area calculation using the continuity equation (Table 4). A total of 2382/32,574 individuals (7.38% 95% CI 7.10 to 7.67%) from the 30% test with a known continuity-derived aortic valve area had severe AS. An output probability cut-off of 0.065425 provided an identical sensitivity and specificity of 91.4% for severe AS diagnosed by AI when compared with the calculated aortic valve area.

### Agreement Between the AI and Continuity-Equation-Derived Severe AS

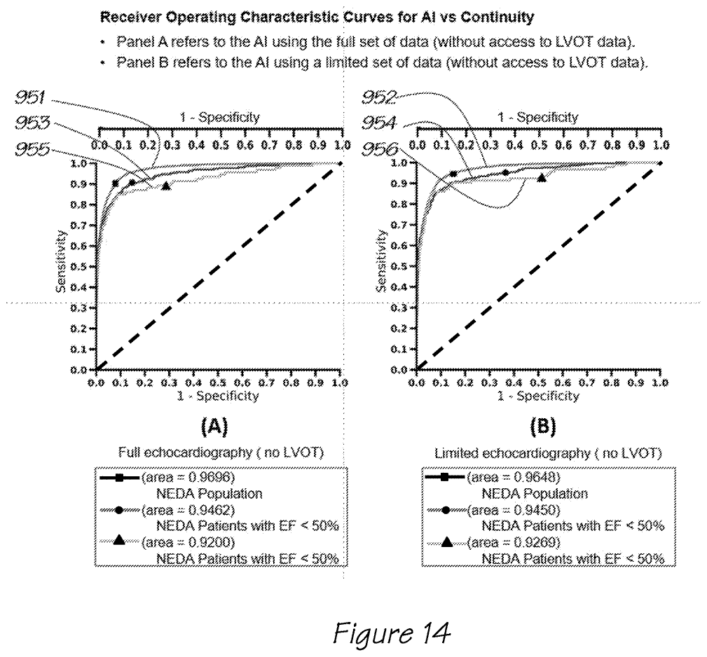

The ROC curve 951 of AI-augmented diagnosis of severe AS compared with that derived from the continuity equation was 0.9696 (see FIG. 14) with a positive predictive value of 45.9%. The model performed almost as well 953 in those with an ejection fraction <50% (FIG. 14) with an AUC of 0.9462 (2308 patients, 11% with severe AS). The AI also performed 955 very well in those with an ejection fraction <30% (491 studies, 13%-many with low gradient, low-output severe AS) with an AUC of 0.9200 (FIG. 14).

In the limited echo cohort, the AI prediction of severe AS remained robust. When applied to the test data set, the AUC of the ROC curve 952 was 0.9648 (FIG. 14). Consistent with the comprehensive data set, the AI performed almost as well in those with an ejection fraction <50% 954 and <30% 956 (AUCs are 0.9450 and 0.9269, respectively-FIG. 14).

### Long Term Survival of Severe AS Diagnosed by AI Versus Continuity Equation

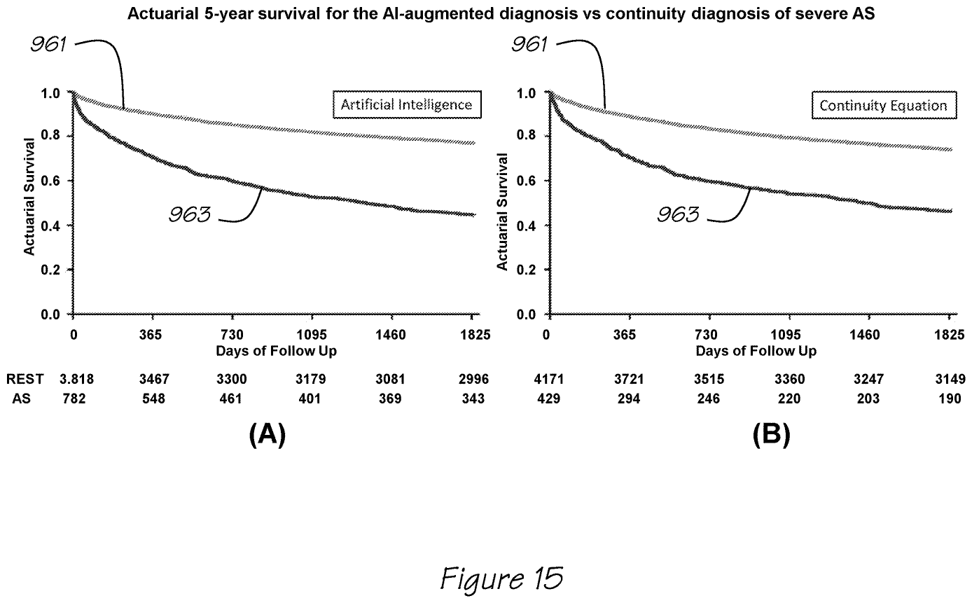

Actuarial 5-year mean (+/−standard error of mean) survival for the AI diagnosis of non-severe AS was 1536.0+/−8.8 days vs 1072.5+/−23.3 days for severe AS, p<0.00001, representing a mean survival difference of 463.5 days (FIG. 15) which shows the actuarial survival curves for diagnosis of severe aortic stenosis using artificial intelligence vs a traditional continuity diagnosis. The patients were matched across the 30% test cohort. The upper line 961 in each of panels (A) and (B) represents the number of individuals at risk without severe aortic stenosis at each time period. The lower line 963 in each of panels (A) and (B) represents number of individuals at risk diagnosed with severe aortic stenosis at each time period. For continuity-derived severe AS, the mean survival was 1489.0+/−8.9 days vs 1086.0+/−31.6 days, a difference of 403 days. The non-severe AS group identified by the AI lived 47 days longer (95% CI 23.3-71.7, p=0.02) than those without severe AS identified by the continuity equation. Alternatively, those with AI-identified severe AS had a similar lifespan to those identified with continuity-derived severe AS (13.5 days, 95% CI-90.4-63.4 days, p=0.7). The AI diagnosis remained highly predictive of death after adjustment for age and gender (adjusted HR=1.37, 95% CI 1.22-1.54, p<0.0001), whereas the continuity diagnosis was only weakly predictive after adjustment for age and gender (HR=1.19, 95% CI 1.04 to 1.37, p=0.025). On an adjusted basis, the AI continued to predict risk of future death (HR=1.34, 95% CI 1.13 to 1.59; p=0.001), consistent with a multi-parameter approach to an AI diagnosis, not just the transaortic valve gradients. Adjusting for clinical outcome such as aortic valve replacement did not significantly change the predictive value of either the AI or continuity equation (CE) above.

### Example 2—Discussion

As demonstrated in Example 2 discussed above, AI can robustly augment the diagnosis of severe AS by interpreting the entire echocardiographic phenotype without reliance on left ventricular outflow tract measurements in a very large cohort of individuals subject to prolonged follow-up. Moreover, consistent with its multi-parameter approach to interpretation, an AI-augmented diagnosis of severe AS remained a significant predictor of long-term mortality even after adjustment for traditional AS severity measures. Accordingly, the purpose-built AI systems disclosed herein also introduce the first potential quality system for echocardiography by providing automatic measurement and disease predictions in real-time. These disclosed systems can provide a known statistical outcome for a defined set of measurements. Importantly, the fully trained AI system takes minimal computing power to operate and can be installed on both echocardiography machines and imaging reading software to improve diagnostic consistency in the absence of expert review.

If proven valid and reliable AI offers an extremely useful clinical tool; particularly in low-resource settings where specialist cardiologists are scarce. Currently, the complex interactions present in AS require evaluation by a subspecialist-trained echocardiographer. Moreover, even after expert review, under-diagnosis of severe AS may occur in some individuals¹⁶⁵. Despite well-developed quality guidelines for diagnosis of AS^{1,3} rigorous application may not be routinely practiced¹⁶⁻¹⁸ and errors may not be identified. The AI evaluated in this study consistently examines the entire echocardiography phenotype; taking into account the known pathophysiologic changes¹⁹⁻²² such as left ventricular diastolic and systolic dysfunction, left atrial enlargement and pulmonary hypertension²³. Reliance on left ventricular outflow tract measures in the continuity equation introduces potential error²⁴⁻²⁶ and potential mis-classification of AS severity, with implications on follow-up echocardiography and timing of intervention. In contrast to current clinical practice, the AI is consistent, completely removing the need for measurement of left ventricular outflow tract dimension or velocity, relevant to both the diagnosis of AS and consistency and timing of follow up¹⁶.

Outside of guidelines, there is no commonly accepted quality metric for clinical echocardiography²⁷. The rigor applied to echocardiographic interpretation in clinical trials^{28, 29} may not be consistently applied in real-world environments, with implications on outcome and decisions for intervention^{30, 31}. Quality improvement programs decrease error rates¹⁷, universal application requires automation and real-time feedback⁶. AI is ideally suited to this task, since its analysis is consistent and phenotype-based. Critically, the AI systems disclosed herein perform equally well on a comprehensive echocardiogram as with a limited data set that takes only 10 minutes to acquire, with implications on efficiency, consistency and cost when applied in specific scenarios (such as follow-up echocardiography for known AS), but this requires further evaluation.

The AI systems disclosed herein are trained on data from NEDA, a very large echo database linked with mortality. Because of the nature and scope of NEDA, quality control against individual images was not feasible and data obtained from individual laboratories is assumed to be correct. However, systematic bias is unlikely because NEDA is sourced from different tertiary hospital laboratories across Australia rather than a single source. Although the AI accurately identified severe AS, the model needs to be tested across the whole range of aortic valve disease; noting the error for imputation of the aortic valve area was small (see Table 4), with no systematic bias and a mean imputation error (95% CI) of −0.056 (−0.885, 0.664)cm². The real-world nature of NEDA data involves missing data points, and a complete set of echocardiography measurements was not obtained in every patient. It is possible some patients with AS did not have sufficient measurements obtained to be included in the AI evaluation, and testing of the AI system in clinical and reference echo laboratories is needed.

The AI may identify some individuals without severe AS, but with similar cardiac phenotypic changes. However, these patients had a similar mortality trajectory to those with traditional severe AS, highlighting the capability of the AI to identify those at high risk. The systems disclosed herein have not yet been validated in populations outside of Australia, although Australia is a multicultural nation broadly representative of the world's population, with over 300 different ancestries and 28% of the resident population born overseas. Also clinical linkage associated with the data sets used was not available for inclusion in the system validation examples discussed above. Potential contributors to the phenotypic changes such as hypertension or valve intervention may also need to be considered in the clinical context of the individual.

In summary, a prototype AI echocardiography measurement interpretation system has been developed which augments the diagnosis of severe AS by evaluating the entire phenotype. This process completely removes the need for left ventricular outflow tract measurements. The AI performs equally in normal and impaired systolic function, including severe left ventricular dysfunction and accurately predicts future mortality risk independent of AS gradients. The systems disclosed herein also provide real-time feedback during echocardiographic examinations with benefits in efficiency, consistency, and follow-up recommendations. There are important implications on study duration, cost and risk of sonographer injury. Overall, a consistent diagnosis of AS severity requires a shift away from the current practice of fully manual interpretation towards a more automated and objective process. The role of AI in these goals requires rigorous clinical evaluation, in differing patient groups, and with different diseases identified by echocardiography.

The validation examples discussed above strongly indicate that AI can augment and improve the diagnosis of severe AS and associated risk mortality predictions, by analysing the entire echo phenotype and without the need for left ventricular outflow tract measurements. Particularly, the AI systems disclosed herein were able to predict patients at higher mortality risk due to AS, independently of aortic valve gradients. Decisions for intervention in severe aortic stenosis (AS) depend on reliable echocardiographic interpretation. The artificial intelligence (AI) systems disclosed herein are particularly designed to augment the diagnosis of severe AS using echocardiographic measurement data, thus providing a significant input into development of effective patient care outcomes by healthcare professionals.

### Interpretation

### Bus

In the context of this document, the term “bus” and its derivatives, while being described in a preferred embodiment as being a communication bus subsystem for interconnecting various devices including by way of parallel connectivity such as Industry Standard Architecture (ISA), conventional Peripheral Component Interconnect (PCI) and the like or serial connectivity such as PCI Express (PCIe), Serial Advanced Technology Attachment (Serial ATA) and the like, should be construed broadly herein as any system for communicating data.

### In Accordance With

As described herein, ‘in accordance with’ may also mean ‘as a function of’ and is not necessarily limited to the integers specified in relation thereto.

### Composite Items

As described herein, ‘a computer implemented method’ should not necessarily be inferred as being performed by a single computing device such that the steps of the method may be performed by more than one cooperating computing devices.

Similarly objects as used herein such as ‘web server’, ‘server’, ‘client computing device’, ‘computer readable medium’ and the like should not necessarily be construed as being a single object, and may be implemented as a two or more objects in cooperation, such as, for example, a web server being construed as two or more web servers in a server farm cooperating to achieve a desired goal or a computer readable medium being distributed in a composite manner, such as program code being provided on a compact disk activatable by a license key downloadable from a computer network.

### Database

In the context of this document, the term “database” and its derivatives may be used to describe a single database, a set of databases, a system of databases or the like. The system of databases may comprise a set of databases wherein the set of databases may be stored on a single implementation or span across multiple implementations. The term “database” is also not limited to refer to a certain database format rather may refer to any database format. For example, database formats may include MySQL, MySQLi, XML or the like.

### Wireless

The invention may be embodied using devices conforming to other network standards and for other applications, including, for example other WLAN standards and other wireless standards. Applications that can be accommodated include IEEE 802.11 wireless LANs and links, and wireless Ethernet.

In the context of this document, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. In the context of this document, the term “wired” and its derivatives may be used to describe circuits, devices, systems, methods, techniques, communications channels, etc., that may communicate data through the use of modulated electromagnetic radiation through a solid medium. The term does not imply that the associated devices are coupled by electrically conductive wires.

### Processes

Unless specifically stated otherwise, as apparent from the following discussions, it is appreciated that throughout the specification discussions utilizing terms such as “processing”, “computing”, “calculating”, “determining”, “analysing” or the like, refer to the action and/or processes of a computer or computing system, or similar electronic computing device, that manipulate and/or transform data represented as physical, such as electronic, quantities into other data similarly represented as physical quantities.

### Processor

In a similar manner, the term “processor” may refer to any device or portion of a device that processes electronic data, e.g., from registers and/or memory to transform that electronic data into other electronic data that, e.g., may be stored in registers and/or memory. A “computer” or a “computing device” or a “computing machine” or a “computing platform” may include one or more processors.

The methodologies described herein are, in one embodiment, performable by one or more processors that accept computer-readable (also called machine-readable) code containing a set of instructions that when executed by one or more of the processors carry out at least one of the methods described herein. Any processor capable of executing a set of instructions (sequential or otherwise) that specify actions to be taken are included. Thus, one example is a typical processing system that includes one or more processors. The processing system further may include a memory subsystem including main RAM and/or a static RAM, and/or ROM.

### Computer-Readable Medium

Furthermore, a computer-readable carrier medium may form, or be included in a computer program product. A computer program product can be stored on a computer usable carrier medium, the computer program product comprising a computer readable program means for causing a processor to perform a method as described herein.

### Networked or Multiple Processors

In alternative embodiments, the one or more processors operate as a standalone device or may be connected, e.g., networked to other processor(s), in a networked deployment, the one or more processors may operate in the capacity of a server or a client machine in server-client network environment, or as a peer machine in a peer-to-peer or distributed network environment. The one or more processors may form a web appliance, a network router, switch or bridge, or any machine capable of executing a set of instructions (sequential or otherwise) that specify actions to be taken by that machine.

Note that while some diagram(s) only show(s) a single processor and a single memory that carries the computer-readable code, those in the art will understand that many of the components described above are included, but not explicitly shown or described in order not to obscure the inventive aspect. For example, while only a single machine is illustrated, the term “machine” shall also be taken to include any collection of machines that individually or jointly execute a set (or multiple sets) of instructions to perform any one or more of the methodologies discussed herein.

### Additional Embodiments

Thus, one embodiment of each of the methods described herein is in the form of a computer-readable carrier medium carrying a set of instructions, e.g., a computer program that are for execution on one or more processors. Thus, as will be appreciated by those skilled in the art, embodiments of the present invention may be embodied as a method, an apparatus such as a special purpose apparatus, an apparatus such as a data processing system, or a computer-readable carrier medium. The computer-readable carrier medium carries computer readable code including a set of instructions that when executed on one or more processors cause a processor or processors to implement a method. Accordingly, aspects of the present invention may take the form of a method, an entirely hardware embodiment, an entirely software embodiment or an embodiment combining software and hardware aspects. Furthermore, the present invention may take the form of carrier medium (e.g., a computer program product on a computer-readable storage medium) carrying computer-readable program code embodied in the medium.

### Implementation

It will be understood that the steps of methods discussed are performed in one embodiment by an appropriate processor (or processors) of a processing (i.e., computer) system executing instructions (computer-readable code) stored in storage. It will also be understood that the invention is not limited to any particular implementation or programming technique and that the invention may be implemented using any appropriate techniques for implementing the functionality described herein. The invention is not limited to any particular programming language or operating system.

### Means for Carrying Out a Method or Function

Furthermore, some of the embodiments are described herein as a method or combination of elements of a method that can be implemented by a processor or a processor device, computer system, or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

### Connected

Similarly, it is to be noticed that the term connected, when used in the claims, should not be interpreted as being limitative to direct connections only. Thus, the scope of the expression a device A connected to a device B should not be limited to devices or systems wherein an output of device A is directly connected to an input of device B. It means that there exists a path between an output of A and an input of B which may be a path including other devices or means. “Connected” may mean that two or more elements are either in direct physical or electrical contact, or that two or more elements are not in direct contact with each other but yet still co-operate or interact with each other.

### EMBODIMENTS

Reference throughout this specification to “one embodiment”, “an embodiment”, “one arrangement” or “an arrangement” means that a particular feature, structure or characteristic described in connection with the embodiment/arrangement is included in at least one embodiment/arrangement of the present invention. Thus, appearances of the phrases “in one embodiment/arrangement” or “in an embodiment/arrangement” in various places throughout this specification are not necessarily all referring to the same embodiment/arrangement, but may. Furthermore, the particular features, structures or characteristics may be combined in any suitable manner, as would be apparent to one of ordinary skill in the art from this disclosure, in one or more embodiments/arrangements.

Similarly it should be appreciated that in the above description of example embodiments/arrangements of the invention, various features of the invention are sometimes grouped together in a single embodiment/arrangement, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment/arrangement. Thus, the claims following the Detailed Description are hereby expressly incorporated into this Detailed Description, with each claim standing on its own as a separate embodiment/arrangement of this invention.

Furthermore, while some embodiments/arrangements described herein include some but not other features included in other embodiments/arrangements, combinations of features of different embodiments/arrangements are meant to be within the scope of the invention, and form different embodiments/arrangements, as would be understood by those in the art. For example, in the following claims, any of the claimed embodiments/arrangements can be used in any combination.

### Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Comprising and Including

In the claims which follow and in the preceding description of the invention, except where the context requires otherwise due to express language or necessary implication, the word “comprise” or variations such as “comprises” or “comprising” are used in an inclusive sense, i.e., to specify the presence of the stated features but not to preclude the presence or addition of further features in various embodiments of the invention.

Any one of the terms: “including” or “which includes” or “that includes” as used herein is also an open term that also means “including at least” the elements/features that follow the term, but not excluding others. Thus, including is synonymous with and means comprising.

### Scope of Invention

Thus, while there has been described what are believed to be the preferred arrangements of the invention, those skilled in the art will recognize that other and further modifications may be made thereto without departing from the spirit of the invention, and it is intended to claim all such changes and modifications as fall within the scope of the invention. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to the mobile device industries, specifically for methods and systems for distributing digital media via mobile devices.

It will be appreciated that the methods/apparatus/devices/systems described/illustrated above at least substantially provide systems and methods for AI-assisted echocardiography.

The AI-assisted echocardiography systems and methods described herein, and/or shown in the drawings, are presented by way of example only and are not limiting as to the scope of the invention. Unless otherwise specifically stated, individual aspects and components of the systems and methods may be modified, or may have been substituted therefore known equivalents, or as yet unknown substitutes such as may be developed in the future or such as may be found to be acceptable substitutes in the future. The systems and methods may also be modified for a variety of applications while remaining within the scope and spirit of the claimed invention, since the range of potential applications is great, and since it is intended that the present systems and methods be adaptable to many such variations.

### REFERENCES

1. Baumgartner H, Falk V, Bax J J, et al. 2017 ESC/EACTS Guidelines for the management of valvular heart disease. European Heart Journal 2017; 38:2739-91.
2. Badiani S, van Zalen J, Treibel T A, Bhattacharyya S, Moon J C, Lloyd G. Aortic Stenosis, a Left Ventricular Disease: Insights from Advanced Imaging. Current Cardiology Reports 2016; 18:80-.
3. Nishimura R A, Otto C M, Bonow R O, et al. 2014 AHA/ACC Guideline for the Management of Patients With Valvular Heart Disease: A Report of the American College of Cardiology/American Heart Association Task Force on Practice Guidelines. Journal of the American College of Cardiology 2014; 63:e57-e185.
4. Mo Y, Penicka M, Di Gioia G, et al. Resolving Apparent Inconsistencies Between Area, Flow, and Gradient Measurements in Patients With Aortic Valve Stenosis and Preserved Left Ventricular Ejection Fraction. The American Journal Of Cardiology 2018; 121:751-7.
5. Bennet C S, Abeya F C, Hoffman A, et al. Performance and Interpretation Training of Transthoracic Echocardiography in Resource-Limited Settings. Journal Of The American College Of Cardiology 2017; 70:1940-1.
6. Pellikka P A, Douglas P S, Miller J G, et al. American Society of Echocardiography Cardiovascular Technology and Research Summit: a roadmap for 2020. Journal Of The American Society Of Echocardiography: Official Publication Of The American Society Of Echocardiography 2013; 26:325-38.
7. Gandhi S, Mosleh W, Shen J, Chow C-M. Automation, machine learning, and artificial intelligence in echocardiography: A brave new world. Echocardiography (Mount Kisco, NY) 2018.
8. Narula S, Shameer K, Salem Omar A M, Dudley J T, Sengupta P P. Machine-Learning Algorithms to Automate Morphological and Functional Assessments in 2D Echocardiography. Journal Of The American College Of Cardiology 2016; 68:2287-95.
9. Zhang J, Gajala S, Agrawal P, et al. Fully Automated Echocardiogram Interpretation in Clinical Practice. Circulation 2018; 138:1623-35.
10. Bishop C. Mixture density networks. Technical Report. Birmingham, UK: Aston University; 1994.
11. Srivastava N H, G; Krizhevsky, A; Sutskever, I; Salakhutdinov, R. Dropout: A Simple Way to Prevent Neural Networks from Overfitting. Journal of Machine Learning Research 2014; 15:1929-58.
12. Grimit E P, Gneiting T, Berrocal V J, Johnson N A. The continuous ranked probability score for circular variables and its application to mesoscale forecast ensemble verification. Quarterly Journal of the Royal Meteorological Society 2007; 132:2925-42.
13. D'Isanto A. Uncertain Photometric Redshifts with Deep Learning Methods. Proceedings of the International Astronomical Union 2016; 12:209-12.
14. Baumgartner H, Hung J, Bermejo J, et al. Recommendations on the Echocardiographic Assessment of Aortic Valve Stenosis: A Focused Update from the European Association of Cardiovascular Imaging and the American Society of Echocardiography. Journal of the American Society of Echocardiography 2017; 30:372-92.
15. Malouf J, Le Tourneau T, Pellikka P, et al. Aortic valve stenosis in community medical practice: determinants of outcome and implications for aortic valve replacement. The Journal Of Thoracic And Cardiovascular Surgery 2012; 144:1421-7.
16. Chan R H, Shaw J L, Hauser T H, Markson L J, Manning W J. Guideline Adherence for Echocardiographic Follow-Up in Outpatients with at Least Moderate Valvular Disease. Journal Of The American Society Of Echocardiography: Official Publication Of The American Society Of Echocardiography 2015; 28:795-801.
17. Fanari Z, Choudhry U I, Reddy V K, et al. The Value of Quality Improvement Process in the Detection and Correction of Common Errors in Echocardiographic Hemodynamic Parameters in a Busy Echocardiography Laboratory. Echocardiography (Mount Kisco, NY) 2015; 32:1778-89.
18. Nagueh S F, Farrell M B, Bremer M L, Dunsiger S I, Gorman B L, Tilkemeier P L. Predictors of Delayed Accreditation of Echocardiography Laboratories: An Analysis of the Intersocietal Accreditation Commission Database. Journal Of The American Society Of Echocardiography: Official Publication Of The American Society Of Echocardiography 2015; 28:1062-9.e7.
19. Delaye J, Chevalier P, Delahaye F, Didier B. Valvular aortic stenosis and coronary atherosclerosis: pathophysiology and clinical consequences. European Heart Journal 1988; 9 Suppl E: 83-6.
20. Pibarot P, Dumesnil J G. New concepts in valvular hemodynamics: implications for diagnosis and treatment of aortic stenosis. The Canadian Journal Of Cardiology 2007; 23 Suppl B: 40B-7B.
21. Dweck M R, Boon N A, Newby D E. Calcific aortic stenosis: a disease of the valve and the myocardium. Journal Of The American College Of Cardiology 2012; 60:1854-63.
22. Mutlak D, Aronson D, Carasso S, Lessick J, Reisner S A, Agmon Y. Frequency, determinants and outcome of pulmonary hypertension in patients with aortic valve stenosis. The American Journal Of The Medical Sciences 2012; 343:397-401.
23. Bartel T, Müller S. Preserved ejection fraction can accompany low gradient severe aortic stenosis: impact of pathophysiology on diagnostic imaging. European Heart Journal 2013; 34:1862-3.
24. Michelena H I, Margaryan E, Miller F A, et al. Inconsistent echocardiographic grading of aortic stenosis: is the left ventricular outflow tract important? Heart (British Cardiac Society) 2013; 99:921-31.
25. Oh J K, Kane G C. The Echo Manual: Lippincott Williams & Wilkins, Lippincott Williams & Wilkins, Attn: Sharon Kimmel, 14700 Citicorp Dr, Bldg 3, Hagerstown, MD, 21742; 2018.
26. Otto C M, Otto C M. The practice of clinical echocardiography: Elsevier Science Health Science, Elsevier Science Health Science, Attn Customer Service, 11830 Westline Industrial Dr, Saint Louis, MO, 63146; 2016.
27. Crowley A L, Yow E, Barnhart H X, et al. Critical Review of Current Approaches for Echocardiographic Reproducibility and Reliability Assessment in Clinical Research. Journal of the American Society of Echocardiography 2016; 29:1144-54.e7.
28. Douglas P S, DeCara J M, Devereux R B, et al. Echocardiographic Imaging in Clinical Trials: American Society of Echocardiography Standards for Echocardiography Core Laboratories. Journal of the American Society of Echocardiography 2009; 22:755-65.
29. Khouri M G, Ky B, Dunn G, et al. Echocardiography Core Laboratory Reproducibility of Cardiac Safety Assessments in Cardio-Oncology. Journal Of The American Society Of Echocardiography: Official Publication Of The American Society Of Echocardiography 2018; 31:361-71.e3.
30. Khouri M G, Ky B, Dunn G, et al. Echocardiography Core Laboratory Reproducibility of Cardiac Safety Assessments in Cardio-Oncology. Journal of the American Society of Echocardiography 2018; 31:361-71.e3.
31. Rigolin V H. Quality Matters. Journal of the American Society of Echocardiography 2017; 30: A17-A8

1. A method for processing a sparsely populated data source comprising:
(a) retrieving data, from a sparsely populated data source, to form a base dataset, the data source comprising a plurality of patient records including patient mortality data, wherein each patient record comprises at least one unpopulated data field corresponding to a medical measurement;
(b) dividing the base dataset into two portions:
a first portion comprising a training dataset being a defined percentage, X %, of the base dataset; and
a second portion comprising a validation dataset being a defined percentage (100%-X %) of the base dataset;
(c) analysing the training dataset to jointly model variable relationships using a non-linear function approximation algorithm applied iteratively to the records of the training dataset to obtain a trained model and measurement prediction protocols for populating unpopulated fields in the training dataset;
(d) using the measurement prediction protocols, computing predicted measurement values for each of the unpopulated data fields;
(e) imputing the predicted measurement values in the patient records of the training dataset;
(f) analysing the training dataset based on at least one predefined disease conditions in a known portion of the patient records of the base dataset to form a phenotype model configured to associate patient phenotype data to a probability of a disease condition in the patient records of a trained dataset;
(g) imputing the predicted measurement values in the records of the validation dataset;
(h) validating the phenotype model, where the validating comprises analysing the validation dataset using the phenotype model, wherein the records of the validation dataset comprise phenotype data associated with patient data, and determining a validation error comprising a probability of correctly predicting the patient phenotype associated with a disease state probability in the records of the validation dataset;
(i) repeating Steps (c) to (h) to minimise the validation error and computing a prediction of a probable disease state phenotype for each patient record in the base dataset.

2. The method of claim 1, wherein analysing the training data set is performed using a using a machine learning system.

3. The method of claim 1, wherein the disease state is aortic stenosis.

4. The method of claim 1, wherein said sparsely populated data source comprises a plurality of medical records comprising measurement data obtained from a medical study procedure.

5. The method of claim 4, wherein the medical study procedure comprises an echocardiography procedure.

6. The method of claim 1, wherein during a measurement procedure, unpopulated measurement data is predicted using the measurement prediction protocols on the basis of data collected by a procedure operator.

7. The method of claim 1, wherein during a measurement procedure, a patient phenotype is determined by the phenotype model based data collected by at least one of: a procedure operator and on measurement data predicted using the measurement prediction protocols.

8. The method of claim 6, wherein the unpopulated measurement data and the patient phenotype is computed in real-time during the measurement procedure.

9. The method of claim 2, wherein the machine learning system comprises:
a neural network;
wherein, during a measurement procedure, measurements obtained by a procedure operator are incorporated into the training data set to form an updated training dataset and analysing the updated training data set using the neural network to compute at least one of: updated measurement prediction protocols and an updated phenotype model; and
wherein, the measurements obtained during the measurement procedure are analysed using at least one of: the updated measurement prediction protocols and the updated phenotype model, to predict a probable disease state for a patient undergoing the measurement procedure.

10. The method of claim 9, further comprising:
as a result of the updated phenotype model associated with the probable disease state, directing a measurement operator to record relevant measurement data to increase the confidence of the patient phenotype and prediction of an associated disease state.

11. A system for conducting a measurement procedure on a patient, the system comprising:
at least one measurement tools configured to perform the measurement procedure;
at least one recording device configured to record measurement data from the patient during the measurement procedure; and
at least one transmitter configured to transmit the measurement data to at least one processor, wherein the processor is configured to:
receive the measurement data and phenotype data;
associate the measurement data and phenotype data to determine a patient phenotype associated with one or more disease states;
generate at least one of:
a measurement prediction protocol configured to predict measurement data for unpopulated measurement fields; and
a phenotype model configured to associate the patient data with a phenotype associated with one or more disease state,
predict a probable disease state for the patient undergoing the measurement procedure; and
alert a measurement operator of the predicted measurement data and the probable disease state; and
provide a set of directions to the measurement operator to collect a dataset of relevant measurement data to be collected based on the predicted probable disease state.

12. The system of claim 11, wherein the disease state is aortic stenosis.

13. The system of claim 11, further comprising a display surface configured to display a notification to the measurement operator comprising the predicted measurement data or a probable disease state.

14. A non-transitory computer readable medium that stores a set of instructions that is executable by at least one processor of a computing device to cause the computing device to perform operations for generating an inspection tool sampling plan, the operations comprising:
(a) retrieving data, from a sparsely populated data source, to form a base dataset, the data source comprising a plurality of patient records including patient mortality data, wherein each patient record comprises_at least one unpopulated data field corresponding to a medical measurement;
(b) dividing the base dataset into two portions:
a first portion comprising a training dataset being a defined percentage, X %, of the base dataset; and
a second portion comprising a validation dataset being a defined percentage (100%-X %) of the base dataset;
(c) analysing the training dataset to jointly model variable relationships using a non-linear function approximation algorithm applied iteratively to the records of the training dataset to obtain a trained model and measurement prediction protocols for populating unpopulated fields in the training dataset;
(d) using the measurement prediction protocols, computing predicted measurement values for each of the unpopulated data fields;
(e) imputing the predicted measurement values in the patient records of the training dataset;
(f) analysing the training dataset based on at least one predefined disease conditions in a known portion of the patient records of the base dataset to form a phenotype model configured to associate patient phenotype data to a probability of a disease condition in the patient records of a trained dataset;
(g) imputing the predicted measurement values in the records of the validation dataset;
(h) validating the phenotype model, where the validating comprises analysing the validation dataset using the phenotype model, wherein the records of the validation dataset comprise phenotype data associated with patient data, and determining a validation error comprising a probability of correctly predicting the patient phenotype associated with a disease state probability in the records of the validation dataset;
(i) repeating Steps (c) to (h) to minimise the validation error and computing a prediction of a probable disease state phenotype for each patient record in the base dataset.

15.-16. (canceled)

17. The non-transitory computer readable medium of claim 14, wherein the disease state is aortic stenosis.
